SEQUENCE LISTING (18 sequences)

SEQ ID NO: 1 - protein, 2214 aa, Homo sapiens
  MISC_FEATURE (1)..(2214): Homo sapiens SorLA Polypeptide
MATRSSRRESRLPFLFTLVALLPPGALCEVWTQRLHGGSAPLPQDRGFLVVQGDPRELRLWARGDARGASRADEKPLRRKRSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 2 - protein, 53 aa, Homo sapiens
  MISC_FEATURE (1)..(53): Homo sapiens SorLA Propeptide
EVWTQRLHGGSAPLPQDRGFLVVQGDPRELRLWARGDARGASRADEKPLRRKR

SEQ ID NO: 3 - protein, 676 aa, Homo sapiens
  MISC_FEATURE (1)..(676): Homo sapiens SorLA Vps10p-Domain
SAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEE

SEQ ID NO: 4 - protein, 465 aa, Homo sapiens
  MISC_FEATURE (1)..(465): Homo sapiens GFRalpha1 Polypeptide Isoform A
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 5 - protein, 120 aa, Homo sapiens
  MISC_FEATURE (1)..(120): Homo sapiens GFRalpha1 Polypeptide Isoform A, Theoretical Binding Site to SorLA
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQ

SEQ ID NO: 6 - protein, 460 aa, Homo sapiens
  MISC_FEATURE (1)..(460): Homo sapiens GFRalpha1 Polypeptide Isoform B
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 7 - protein, 115 aa, Homo sapiens
  MISC_FEATURE (1)..(115): Homo sapiens GFRalpha1 Polypeptide Isoform B, Theoretical Binding Site to SorLA
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFIS

SEQ ID NO: 8 - protein, 464 aa, Homo sapiens
  MISC_FEATURE (1)..(464): Homo sapiens GFRalpha2 Polypeptide
MILANVFCLFFFLDETLRSLASPSSLQGPELHGWRPPVDCVRANELCAAESNCSSRYRTLRQCLAGRDRNTMLANKECQAALEVLQESPLYDCRCKRGMKKELQCLQIYWSIHLGLTEGEEFYEASPYEPVTSRLSDIFRLASIFSGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 9 - protein, 331 aa, Homo sapiens
  MISC_FEATURE (1)..(331): Homo sapiens GFRalpha2 Polypeptide, Short Isoform
MILANVFCLFFFLGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 10 - protein, 400 aa, Homo sapiens
  MISC_FEATURE (1)..(400): Homo sapiens GFRalpha3 Polypeptide
MVRPLNPRPLPPVVLMLLLLLPPSPLPLAAGDPLPTESRLMNSCLQARRKCQADPTCSAAYHHLDSCTSSISTPLPSEEPSVPADCLEAAQQLRNSSLIGCMCHRRMKNQVACLDIYWTVHRARSLGNYELDVSPYEDTVTSKPWKMNLSKLNMLKPDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 11 - protein, 369 aa, Homo sapiens
  MISC_FEATURE (1)..(369): Homo sapiens GFRalpha3 Polypeptide, Isoform 2
MVRPLNPRPLPPVVLMLLLLLPPSPLPLAAGDPLPTESRLMNSCLQARRKCQADPTCSAAYHHLDSCTSSISTPLPSEEPSVPADCLEAAQQLRNSSLIGCMCHRRMKNQVACLDIYWTVHRARSLDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 12 - protein, 299 aa, Homo sapiens
  MISC_FEATURE (1)..(299): Homo sapiens GFRalpha4 Polypeptide
MVRCLGPALLLLLLLGSASSVGGNRCVDAAEACTADARCQRLRSEYVAQCLGRAAQGGCPRARCRRALRRFFARGPPALTHALLFCPCAGPACAERRRQTFVPSCAFSGPGPAPPSCLEPLNFCERSRVCRCARAAAGPWRGWGRGLSPAHRPPAAQASPPGLSGLVHPSAQRPRRLPAGPGRPLPARLRGPRGVPAGTAVTPNYVDNVSARVAPWCDCGASGNRREDCEAFRGLFTRNRCLDGAIQAFASGWPPVLLDQLNPQGDPEHSLLQVSSTGRALERRSLLSILPVLALPALL

SEQ ID NO: 13 - protein, 13 aa, Homo sapiens
  MISC_FEATURE (1)..(13): Homo sapiens Neurotensin Polypeptide
QLYENKPRRPYIL

SEQ ID NO: 14 - protein, 3 aa, Homo sapiens
  MISC_FEATURE (1)..(3): Homosapiens Neurotensin Polypeptide Theoretical Binding Site
YIL

SEQ ID NO: 15 - protein, 4 aa, Homo sapiens
  MISC_FEATURE (1)..(4): Homo sapiens Neurotensin Polypeptide Theoretical Binding Site
PYIL

SEQ ID NO: 16 - protein, 211 aa, Homo sapiens
  MISC_FEATURE (1)..(211): Homo sapiens GDNF Polypeptide
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 17 - protein, 58 aa, Homo sapiens
  MISC_FEATURE (1)..(58): Homo sapiens GDNF Polypeptide, Propeptide
FPLPAGKRPPEAPAEDRSLGRRRAPFALSSDSNMPEDYPDQFDDVMDFIQATIKRLKR

SEQ ID NO: 18 - protein, 38 aa, Homo sapiens
  MISC_FEATURE (1)..(38): Homo sapiens GDNF Polypeptide, Binding Site
SPDKQMAVLPRRERNRQAAAANPENSRGKGRRGQRGKN

(19) United States
(12) Patent Application Publication
(10) Pub. No.: US 2015/0239973 A1
(43) Pub. Date: Aug. 27, 2015

(54) Modulation of the Interaction Between SorLA and GDNF-Family Ligand Receptors

(71) Applicant: H. LUNDBECK A/S, Valby (DK)

(72) Inventors: Anders Nykjær, Risskov (DK); Simon Glerup, Risskov (DK); Peder Søndergaard Madsen, Risskov (DK); Claus Munck Petersen, Arhus C (DK); Ditte Olsen, Arhus V (DK)

(73) Assignee: H. LUNDBECK A/S, Valby (DK)

(21) Appl. No.: 14/681,155

(22) Filed: Apr. 8, 2015

Related U.S. Application Data
(63) Continuation of application No. 13/702,692, filed on Jan. 16, 2013.
(60) Provisional application No. 61/354,536, filed on Jun. 14, 2010.

(30) Foreign Application Priority Data
Jun. 14, 2010 (DK) PA201000522

(51) Int. Cl. C07K 16/28

(52) U.S. Cl. CPC C07K 16/286; C07K 2317/76; C07K 2317/24; C07K 2317/622; C07K 2317/21; A61K 2039/505

(57) ABSTRACT

The present invention relates to a method to increase the survival of neurons by modulating the interaction between Sor LA and GDNF-family ligand receptors. The agent used to modulate the interaction between the SorLA and GDNF-family ligand receptors are selected from proteins, peptides, antibodies or small organic compounds. The invention also relates to a pharmaceutical compositions comprising these agent as well as the use of said agent or pharmaceutical composition in the treatment of a disease associated with the loss of neurons and/or wherein the survival of neurons are desired.

### CROSS-REFERENCE TO RELATED APPLICATIONS

The present application claims priority to U.S. patent application Ser. No. 13/702,692 (filed on Jan. 16, 2013; pending), which application is a §371 National Stage of PCT International Patent Application No. PCT/DK2011/050214 (filed on Jun. 14, 2011; expired), which application claims priority to U.S. Patent Application No. 61/354,536 (filed Jun. 14, 2010; lapsed) and DK application PA201000522 (filed Jun. 14, 2010), each of which applications is herein incorporated by reference in its entirety.

### REFERENCE TO SEQUENCE LISTING

This application includes one or more Sequence Listings pursuant to 37 C.F.R. 1.821 et seq., which are disclosed in both paper and computer-readable media, and which paper and computer-readable disclosures are herein incorporated by reference in their entirety

### FIELD OF THE INVENTION

The present invention relates to the modulation of the Vps10p-domain receptor SorLA and its interactions with GDNF-family ligand receptors. Agents that are able to modulate or inhibit this interaction have a potential in the treatment of neurological, mental, and behavioral disorders. The invention provides such ligands capable of acting as modulators of signaling via SorLA in addition to but not limited to the retrograde transport and sorting of GDNF. The agents are thus selected from antagonists/inhibitors and agonists depending on the specific type of neurological, mental and behavioral disorder. The present invention also relates to assays for identifying new agents that can modulate interaction between SorLA and the GDNF-family ligand receptors.

### BACKGROUND OF THE INVENTION

Diseases of the nervous system and in particular mental and behavioural disorders are among the leading causes of disability, accounting for more than 37 percent of years of life lived with disability amongst adults aged 15 years and older worldwide, and as illness likely to represent an increasingly greater health, societal and economic problem in the coming years (Lopez and Murray 1998). Diseases of the nervous system are defined in e.g. ICD10, Chapter VI, Blocks GOO-G99 (Diseases of the nervous system) from World Health Organization, and include for example neurodegenerative diseases such as Parkinson's disease. Mental and behavioral disorders are defined in e.g. ICD10, Chapter V, Blocks FOO-F99 (Mental and behavioral disorders) from World Health Organization, and includes for example major depressive disorders, schizophrenia, attention deficit and hyperactivity disorder (ADHD), drug abuse, anxiety disorders, and bipolar disorder (manic depressive illness). These disorders are common, severe, chronic, and often life-threatening illness. Suicide is estimated to be the cause of the death in up to 15% of the individuals with disorders such as major depressive disorders and bipolar disorder, and many other deleterious health-related effects have been recognized (Michelson, Stratakis et al. 1996; Musselman, Evans et al. 1998; Ciechanowski, Katon et al. 2000; Schulz, Beach et al. 2000; Kupfer 2005). It is increasingly being recognized that some of these disorders are systemic diseases with deleterious effects on multiple organ systems.

Altered neuronal activity, survival, and in particular impairment in synaptic plasticity and transmission is believed to underlie the pathophysiology of neuronal disease. The neurotransmitter dopamine, in particular, is involved in several common disorders of brain function, notably Parkinson's disease, schizophrenia, attention deficit and hyperactivity disorder, as well as in drug dependence and certain endocrine disorders. Many of the drugs clinically used to treat these conditions work by influencing dopamine transmission. There are three main dopaminergic pathways. The nigrostriatal pathway that is important in motor control and the selective degeneration of the dopaminergic neurons of this pathway is a hallmark of Parkinson's disease. The mesolimbic/mesocortical pathways are running from groups of cells in the midbrain to parts of the limbic system, especially the nucleus accumbens, and to the cortex; they are involved in emotion and drug-induced reward systems. Finally, the tuberohypophyseal pathway running from the hypothalamus to the pituitary gland, the secretions of which they regulate.

Neurotrophic factors are potent mediators of neuronal “stay alive” signals and have profound effect on synaptic transmission and plasticity. Glia cell-derived neurotrophic factor (GDNF) is one of a group of related homodimeric neurotrophic factors that also includes neuturin (NRTN), persephin (PSPN) and artemin (ARTN). Together, these are denoted GDNF-family ligands (GFL). GDNF is expressed throughout the developing nervous system, but in the adult brain it is particularly highly expressed in neurons of striatum, thalamus, cortex and hippocampus. Due to retrograde transport, a significant amount of GDNF, originating from striatum, is furthermore found in the substantia nigra. Expression is also explicit astrocytes and Schwann cells as well as in non-neuronal tissues like kidney, testis, and skeletal muscle.

GDNF has been functionally associated with Parkinson's disease (Lin et al, 1993). Parkinson's disease is characterized by a progressive loss of dopaminergic neurons of the substantia nigra and the subsequent loss of dopaminergic neuronal innervation of the striatum. This pathway is essential for voluntary motor behavior. As the nigrostriatal dopaminergic pathway selectively degenerates, dopamine in basal ganglia relapses. The cardinal locomoter symptoms, i.e. tremor, akinesia, rigidity, postural instability, and bradykinesia manifest itself when neuronal losses exceed more than 50-60%. Most of these motor symptoms can almost be completely reversed by dopamine replacement (Sian et al, 1999). In a study of the distribution of neurotrophic factors in substantia nigra of postmortem human Parkinson's disease brains, NGF, NT-3, and NT-4 showed little or no change, while GDNF, BDNF, and CNTF showed significant reductions in Parkinson's disease brains compared to age-matched controls; with GDNF being considerably more depleted (Chauhan et al, 2001). The study focused on neuronal levels and concentrations in neuropil, i.e. the dense interstice among the neurons in the gray matter consisting of interwoven axons, dendrites and glial cells. Notably, GDNF was extensively diminished in both regions, but relatively more in the neuropil. The molecular etiology of Parkinson's disease is not fully understood, but GDNF seems to be implicated.

GDNF signalling is mediated via its interaction with two receptors. The active dimer first binds its primary receptor GFRα1, which serves to concentrate the ligand on the cell membrane, and the resulting tetrameric (2:2) complex subsequently interact with the homodimeric receptor tyrosine kinase RET to induce RET phosphorylation and form a signalling complex. Neuturin, persephin and artemin uses a similar mechanism for signal-transduction, but each of the three binds a separate type (2-4) of GFRα before targeting the Ret receptor. GFRα receptors are linked to the plasma membrane through a glycosylphosphatidylinositol (GPI) anchor but can be released from the cell surface by an unknown phospholipase or proteinase. Soluble GFRα1 can also stimulate RET phosphorylation but the downstream signalling pathways initiated by GDNF together with membrane-bound or soluble GFRα1 are different.

Along with its relatives, GDNF plays an important role in the survival of motor neurons, in the development of sympathetic and sensory neurons, and in hippocampal synaptogenesis. It promotes survival and re-growth of dopaminergic neurons after adult brain injury and is essential to the maintenance and survival of adult dopamine neurons. Thus, GDNF is an attractive target for treatment Parkinson's disease. Nevertheless, GDNF, GFRα1, and Ret are not required for the development of the dopaminergic system during embryogenesis. Instead, genetic disruption of GDNF or receptors results in accelerated degeneration of the nigrostriatal system in aging mice. Also, no genetic association have been drawn between Parkinson's disease and the GDNF system, whereas several reports have suggested genetic linkage between GDNF signalling and the development of attention-deficits and hyperactivity disorder (ADHD) (Syed et al. 2007 Am J Med Genet), and schizophrenia (Souza et al. 2010 J Psychiatr Res)(Williams et al. 2007 Schizophr Res). Interestingly, potentiation of GDNF signalling in the ventral tegmental area and the nucleus accumbens decreases the response and sensitization to psychostimulants such as cocaine in rodents, suggesting that GDNF plays a critical role for the behavioural response to drugs of abuse. Although Ret is the established GDNF signalling receptor, many cells expressing GFRα1 do not express Ret, suggesting the existence of alternative GDNF/GFRα1 receptors.

RET, GFRα1, and GDNF knock-out mice are reported to share strikingly similar phenotypes, likely a consequence of the three proteins participating in the same signaling cascade. In fact, mice deficient in GDNF (Moore et al, 1996), GFRα1 (Cacalano et al, 1998; Enomoto et al, 1998; Tomac et al, 2000) or Ret (Durbec et al, 1996; Schuchardt et al, 1994) all die postnatally due to kidney agenesis and lack of several parasympathetic neurons as well as the entire enteric nervous system. Surprisingly, mesencephalic dopaminergic neurons in addition to numerous other neuronal populations in PNS and CNS of the GFRα1 knock-out mouse display no or few abnormalities at birth (Tomac et al, 2000). From these knock-out reports, GDNF, GFRα1, and RET seems to be uninvolved in neuronal development in general, although neuronal lineages derived from the neuronal crest, e.g. most of the enteric nervous system and the superior cervical ganglion, dependent on functional Ret for normal development, as these neurons are reportedly absent in knock-out embryos and neonates (Durbec et al, 1996). As knock-out mice die shortly after birth, the CNS long-term consequences of depletion are unevaluated. Other approaches are applied to study the consequences during infancy and adulthood. Heterozygous GFRα1+/−mice are viable to adulthood, and show a decreased GDNF-mediated neuroprotection compared to wild type littermates, demonstrated by induction of cerebral ischemia. The level of accessible GFRα1 is thus suggested to be the limiting factor in GDNF efficiency (Tomac et al, 2000). Elaboration on the GFRα1+/−heterozygote mouse revealed a reduction in dopamine in the striatum, and an age-dependent decrease in dopaminergic neurons of the substantia nigra accompanying diminished motor activity (Zaman et al, 2008). Regionally selective RET ablation leads to progressive loss of dopaminergic neurons specifically in the substantia nigra of senescent mice (Kramer et al, 2007). Loss of nigrostriatal innervations indirectly affects dopaminergic striatal neurons, leading to substantial age-dependent degradation of nerve terminals in striatum and reduced levels of evoked dopamine release. RET is thus critical to maintenance of the nigrostriatal dopamine system (Kramer et al, 2007). Equally, adult wild type mice engrafted with fetal neural GDNF−/− tissue in the midbrain, are utilized in the study of continued postnatal development of mesencephalic dopaminergic neurons with GDNF null mutation (Granholm et al, 2000). The GDNF−/− tissue engraftment resulted in reduced number of dopaminergic neurons and neurite outgrowth, and the study demonstrated that GDNF is critical for the long-term survival of mesencephalic dopaminergic neurons (Granholm et al, 2000). The age-dependent decline in the nigristriatal dopaminergic system function is general to the GDNF signaling pathway-impaired mice, and this phenotype is similar to the alterations associated with the early phases of Parkinson's disease.

Indication of the GFL-GFRα-RET cohesiveness is the complementary expression pattern in CNS of adult mice. Here, GDNF and NRTN responsive brain regions expressing RET, either co-express GFRα1 or GFRα2. Vice versa, several regions receiving projections from GFRα1 or GFRα2 expressing neurons have endogenous GDNF and NRTN (Golden et al, 1998). The pairing is specific, as GFL and corresponding GFRα knock-out mice exhibit very similar phenotypes (Airaksinen et al, 1999). However regarding neurons of the superior cervical sympathetic ganglia, the phenotypes of GDNF−/−, GFRα1−/−, and RET−/− mice are conflicting. These neurons are completely depleted in RET null mice (Durbec et al, 1996); while they are only affected to a minor extent (35% reduction in neuron number) in GDNF−/− neonates (Moore et al, 1996), they appear normal and unaffected in GFRα1 knock-out mice (Enomoto et al, 1998). This could be explained by redundancy among GFLs and GFRαs in these neurons, since superior cervical sympathetic ganglionic neurons depend on ARTN-GFRα3 signaling for formation, migration and postnatal survival (Nishino et al, 1999). Consequently, GFRα3−/− mice are viable and fertile, but display a distinct phenotype of eyelid ptosis. Despite a widespread GFRα3 expression in diverse ganglia, GFRα3-mediated signaling is indecisive in other PNS ganglia (Nishino et al, 1999). GFRα2 is an essential factor in development of the several postganglionic parasympathetic neurons, and GFRα2 ablation results in lacrimal and saliva gland malfunctioning in addition to small intestine myenteric plexus (i.e. part of the enteric nervous system controlling gastrointestinal tract motility) deficiency due to failed cholinergic fiber innervations (Rossi et al, 1999). GFRα2−/− mice are, like GFRα3 knock-out mice, viable (Rossi et al, 1999) and display no deficiencies in CNS, even though NRTN and GFRα2 are expressed throughout the adult CNS (Golden et al, 1998). The fact that only minor phenotypes is observed in the CNS of GFL or GFRα knock-out mice may be due to trophic redundancy for central neurons.

Taken together, dysfunction of the signaling induced by GDNF, neuturin, artemin, or persephin is linked functionally or genetically to a number of disorders, notably motor neuron disease, sensory regeneration and neuropathic pain, ischaemia, epilepsy, Parkinson's disease, drug abuse, and schizophrenia.

### SorLA

Sorting protein-related receptor abbreviated SorLA (Swiss prot ID no Q92673), also known as LR11, is a 250-kDa type-1 membrane protein and the second member identified in the Vps10p-domain receptor family. All the receptors in this family share the structural feature of an approximately 600-amino acid N-terminal domain with a strong resemblance to each of the two domains, which constitute the luminal portion of the yeast sorting receptor Vps10p (Marcusson, Horaz-dovsky et al. 1994). The Vps10p-domain (Vps10p-D) that among other ligands binds neurotrophic factors and neuropeptides (Mazella, Zsurger et al. 1998; Munck Petersen, Nielsen et al. 1999; Nykjaer, Lee et al. 2004; Westergaard, Sorensen et al. 2004; Teng, Teng et al. 2005), constitutes the entire luminal part of the first identified member Sortilin and is activated for ligand binding by enzymatic propeptide cleavage (Mazella, Zsurger et al. 1998; Munck Petersen, Nielsen et al. 1999). SorLA, like sortilin, whose lumenal domain consists of a Vps10p domain only, is synthesized as a proreceptor that is cleaved by furin in late Golgi compartments. It has been demonstrated that propeptide cleavage conditions the Vps10p domain for propeptide inhibitable binding of neuropeptides and the receptor-associated protein. The sequence of the SorLA vps10-p domaine is given in SEQ ID NO 3. It has been demonstrated (Jacobsen, Madsen et al. 2001) that avid binding of the receptor-associated protein, apolipoprotein E, and lipoprotein lipase not inhibited by propeptide occurs to sites located in other lumenal domains. In transfected cells, about 10% of full length SorLA is expressed on the cell surface capable mediating endocytosis. The major pool of receptors is found in late Golgi compartments, and interaction with newly synthesized ligands has been suggested. SorLA is highly expressed in distinct cell types throughout the nervous system both during development and in the adult organism (Kanaki, Bujo et al. 1998; Motoi, Aizawa et al. 1999; Offe, Dodson et al. 2006). Interestingly, SorLA levels are reduced in the sporadic form of Alzheimer's disease (Scherzer, Offe et al. 2004; Dodson, Gearing et al. 2006; Sager, Wuu et al. 2007) and inherited mutations in the SorLA gene are genetically linked to late-onset Alzheimer's disease (Rogaeva, Meng et al. 2007). Importantly, SorLA has been shown to mediate high affinity binding and sorting of amyloid precursor protein, and to confer protection against Aβ generation (Andersen, Reiche et al. 2005; Offe, Dodson et al. 2006; Spoelgen, von Arnim et al. 2006; Rogaeva, Meng et al. 2007).

Although a number of drugs are already available for the treatment of neurological, mental and behavioral disorders, all have complex indirect mechanism of action, and are aimed at alleviating the symptoms rather than at treating the underlying cause of the disease. Instead, it is generally believed that drugs that target the signaling pathways that regulate synaptic plasticity or neuronal survival should be developed as long-term treatments for neurological, mental and behavioural disorders (Manji, Drevets et al. 2001). Extensive experimental and clinical data suggest a central function for GDNF signaling in mental and behavioral disorders, and in disorders of the nervous system in general. For example, polymorphisms in the GDNF or GFRα1-3 genes correlate with schizophrenia, and GDNF polymorphisms are associated with attention deficit and hyperactivity disorder. Furthermore, altered GDNF serum levels correlate with bipolar disorder.

Parkinson's disease is a neurodegenerative disorder that is characterized by impairment of motor and cognitive functions due to the progressive death of selected neurons predominantly midbrain dopaminergic neurons within the pars compacta and substantia nigra. This results in a reduced level of dopamine (reviewed in Davie 2008). Current therapy for Parkinson's disease is merely symptomatic treatment, since the underlying neuronal degeneration continues. This palliative care includes administration of dopamine agonists and LDOPA in combination with various inhibitors of dopamine metabolism. GDNF has been shown to possess neuroprotective and neuroregenerative effects on dopaminergic neurons (Beck et al. 1995; Kearns et al. 1995; Lin et al. 1993; Sauer et al. 1995) it has been considered as a treatment for Parkinson's disease. Positive effects of GDNF have been shown in MPTP- and 6-OHDA-lesioned animal models of Parkinson's disease, where GDNF was shown to promote the survival of mesencephalic dopaminergic neurons (Gash et al. 1998; Tomac et al. 1995). Experiments with intracerebral GDNF injection into parkinsonian rhesus monkeys revealed a significant relieve of three cardinal symptoms of Parkinson's disease, i.e. bradykinesia, rigidity, and postural instability (Gash et al, 1996). The GDNF recipients had significant enhanced dopamine levels, fibre density and cell size of the mesencephalic dopaminergic neuron and notably, the number of dopaminergic neurons in substantia nigra was increased. The problem with GDNF treatment is delivery, as GDNF has difficulties penetrating the blood-brain barrier. Gene therapy in parkinsonian rodents and monkeys are tried with positive outcome (Zurn et al, 2001). GDNF delivered by an encapsulated genetically engineered cell line resulted in continued release of low levels of GDNF and subsequent protection of nigral dopaminergic neurons and behavioral recovery. Moreover, GDNF delivery by a lentiviral vector system showed regeneration of the neurons of substantia nigra leading to reversal of the parkinsonian functional deficits. However, results from clinical trials testing GDNF as treatment for Parkinson's disease have had different outcome. Infusing GDNF intraventricular failed to produce symptomatic benefits in Parkinson's disease patients and was associated with a number of adverse side effects including nausea, hallucinations, and depression (Nutt et al. 2003). Administrating GDNF into the striatum instead, was associated with significant clinical benefits (Gill et al. 2003), however, these results could not be confirmed in a double-blinded, placebo-controlled trial (Lang et al. 2006). Although the clinical trials are inconclusive as different results have been obtained, GDNF should still be considered as a potential treatment for Parkinson's disease, but there are several obstacles that must be overcome. These include delivery of GDNF: it needs to cross the blood-brain barrier, GDNF needs to diffuse to the appropriate target and it is necessary to minimize the effect of non target delivery to decrease adverse side effects.

### SUMMARY OF THE INVENTION

The present invention relates to a method to increase the survival of neurons, such as dopaminergic neurons, by modulating the interaction between SorLA and GDNF-family ligand receptors, such as the GFRα1, 2, 3, and/or 4 receptors. According to one embodiment the extracellular levels of GDNF in the brain of a patient in the need thereof may be increased by modulating or inhibiting the interaction between SorLA and GFRα1, or the internalisation and/or the degradation of GDNF is inhibited.

In particular the present invention relates to a method, wherein the agent used to modulate the interaction between the SorLA and GDNF-family ligand receptors is selected from proteins, peptides, antibodies or small organic compounds.

The invention also relates to a pharmaceutical composition comprising such agents in combination with one or more pharmaceutically acceptable carriers or diluents or in combination with an adjuvant.

Additionally the invention relates to the use of such agents or pharmaceutical compositions to increase the survival of neurons, such as e.g. dopaminergic neurons, in particular within the diseases comprising injury induced neural cell death, spinal cord injury, peripheral nerve damage, cerebral ischemia, motor neuron disease, amyotrophic lateral sclerosis, chronic pain, neuropathic pain, epilepsy, cancer, Parkinson's disease, major depressive disorder, schizophrenia, attention deficit and hyperactivity disorder (ADHD), drug abuse, anxiety disorder, and/or bipolar disorder (manic depressive illness).

Finally, the invention relates to various assays for identifying agents that are able to inhibit the interaction between SorLA and the GDNF-family ligand receptors.

### BRIEF DESCRIPTION OF DRAWINGS

FIG. 1. GDNF family ligand signaling

FIG. 2. Domain structure of Vps10p-domain receptors

FIG. 3. SorLA selectively binds GDNF but not other GDNF-family ligands (A) The domain structure of SorLA is depicted. (B) GDNF binds to immobilized SorLA in a concentration-dependent manner as shown by SPR. The calculated KD is in the range of 3-8 nM. (C) SorLA binding is selective for GDNF and not the other GDNF family ligands, artemin, neuturin, and persephin. The neurotrophic factor concentrations used are 20 nM. (D) The interaction is mediated by the mature part of GDNF and not the GDNF propeptide (GDNFpro). Sensorgrams of 100 nM ligand concentration are shown. (E) Excess SorLA propeptide (SorLApro, 10 μM) partially inhibits GDNF binding (100 nM). (F) Binding of GDNF (50 nM) is completely inhibited by excess neurotensin (NT, 20 μM). (G-H) Internalization of 50 nM GDNF by 293 cells or 293 cells stably expressing SorLA. Internalized GDNF (green) was visualized by immunofluorescense. (I) GDNF internalization was inhibited by excess neurotensin (NT, 20 μM).

FIG. 4. Cooperative GDNF uptake by SorLA and GFRα1

(A) GDNF (50 nM) internalized by SorLA during 45 min was visualized by immunofluoerescense. However, internalization could not be detected using 10 nM GDNF. GDNF (50 nM) was not internalized by cells expressing GFRα1 but remained bound to surface. (B) GDNF is internalized to a large extent by cells expressing both SorLA and GFRα1. (C) Time course of the process of GDNF (10 nM) internalization from the surface by SorLA and GFRα1.

FIG. 5. SorLA and GFRα1 forms a cell surface GDNF receptor complex

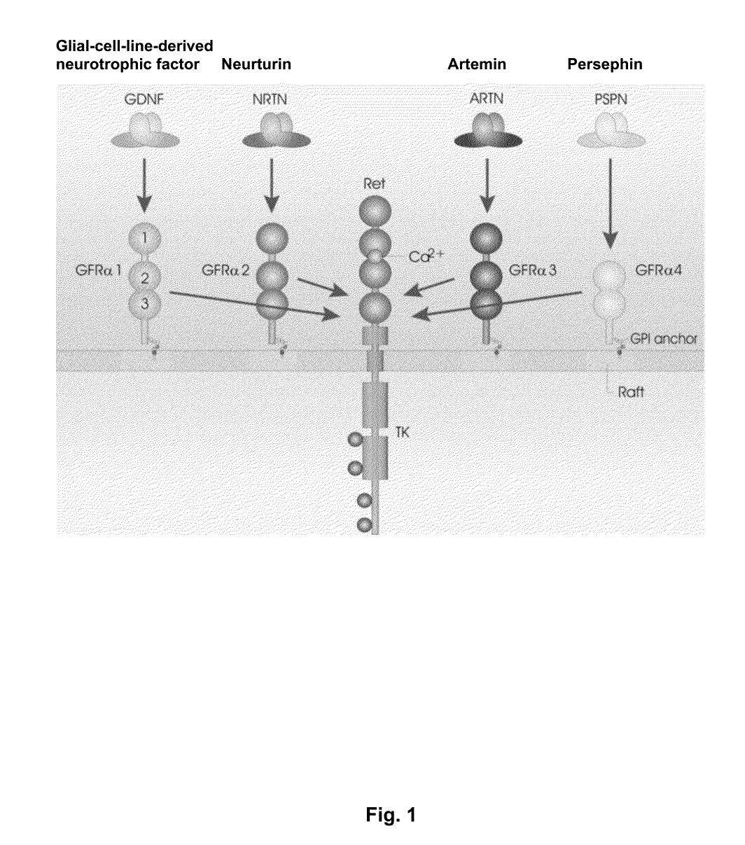

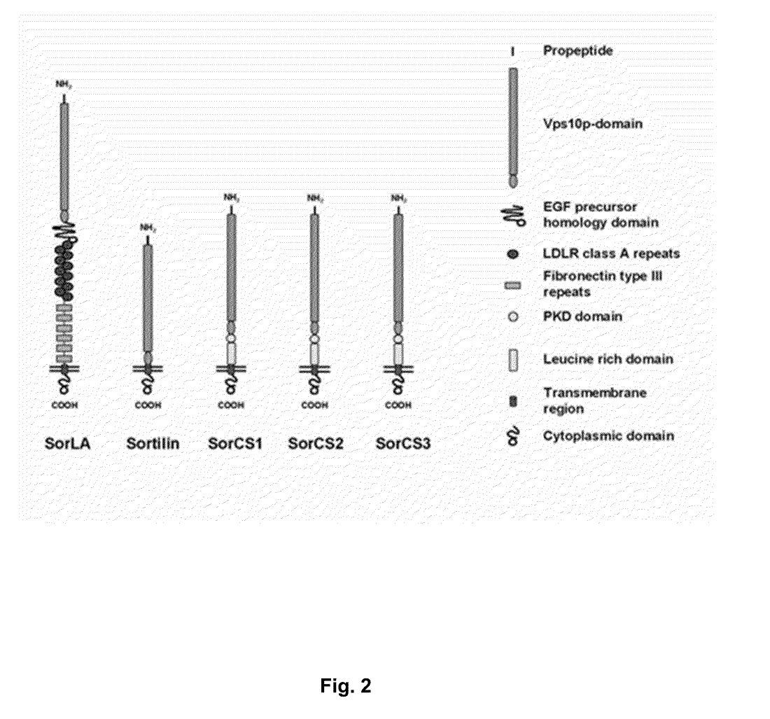

(A) SorLA interacts directly with GFRα1 in cells as shown by co-immunoprecipitation of SorLA with GFRα1 from cells expressing both receptors.

(B) Fluorescence resonance energy transfer (FRET) analysis of the cell surface interaction between SorLA (acceptor) and GFRα1 (donor) in non-permeabilized fixed cells using antibodies against the extracellular domains and fluorescentlylabelled secondary antibodies. A representative cell is shown. (C) Cell surface

SorLA/GFRα1 complex formation is highly specific. Mean Eₐₚₚ% values were calculated within regions of interest (ROI) of size 5×5 pixels in all FRET channel images and are plotted as a function of the corresponding mean acceptor signals of the ROIs. The nearly constant dependence of Eₐₚₚ% on the acceptor signal is indicative of a specific interaction. (D) Histogram showing normal distribution of Eₐₚₚ% values with a mean of 16% for the SorLA/GFRα1 interaction (black histogram). For comparison, the Eₐₚₚ% values were measured for SorLA and Ret in stably transfected cells (grey histogram). The mean Eₐₚₚ% value was 6% suggesting that a potential SorLA/Ret interaction is not specific. (E) GFRα1 binds to the immobilized SorLA extracellular domain in a concentration dependent manner with a KD of approximately 6 nM. (F) Specific interaction of GFRα1 but not Ret extracellular domain (200 nM of either) with SorLA. (G) Unlike the SorLA/GDNF interaction, GFRα1 (200 nM) binding by SorLA is not inhibited by excess neurotensin (NT, 20 μM). (H) GFRα1 (200 nM) binding to SorLA is inhibited by excess SorLA propeptide (5 μM).

FIG. 6. Retrograde sorting of GFRα1/GDNF by SorLA

(A) GFRα1 (green) is internalized from the cell surface over time in the presence of SorLA. (B) The internalization appears to be enhanced by the presence of GDNF. (C) GFRα1 alone is not internalized during the course of the experiment.

(D) The SorLA/GFRα1 interaction is stable at low pH, suggesting the complex persists at endosomal pH. (E) SorLA/GDNF and SorLA/GFRα1 are not competitive interactions as shown by SPR analysis of the binding 50 nM of GDNF and 50 nM GFRα1 to immobilized SorLA.

FIG. 7. SorLA regulates GFRα1 subcellular localization

(A) The presence of SorLA results in a more vesicle-like localization of GFRα1 in stably transfected cells. (B) Surface biotinylation experiments showing a reduction in surface localized GFRα1 in cells expressing SorLA. (C) SorLA decreases the relative amount of GFRα1 in fractions containing flotilin, a lipid raft marker, as assessed by sucrose gradient centrifugation.

FIG. 8. SorLA disrupts Ret surface clustering

(A) Overexpression of SorLA alters the subcellular localization of endogenous Ret in SY5Y neuroblastoma cells as shown by equilibrium gradient centrifugation. (B) Ret stainings of non-permeabilized differentiated SY5Y cells showing that the presence of surface localized Ret clusters disappears when wild-type SorLA is overexpressed but not by overexpression of SorLA deltatail. (C) Immunofluorescense stainings of differentiated neuroblastoma (SY5Y) cells expressing endogenous Ret and some SorLA, overexpressing wild-type SorLA or a C-terminal truncated SorLA variant lacking the cytoplasmic tail (SorLA deltatail). In nontransfected cells, the endogenous Ret is observed in intensely stained dispersed clusters. However, Ret clustering is completely disrupted by SorLA overexpression. Truncation of the SorLA cytoplasmic tail rescues Ret clustering.

FIG. 9. SorLA modulates Ret signaling and downstream functions

(A) Stimulation of SY5Y neuroblastoma cells with 6 nM GDNF+/−6 nM soluble GFRα1 for 0, 15, and 45 min. Ret was immunoprecipitated using anti-Ret antibodies and analyzed by Western blotting using anti-phosphotyrosine antibodies.

(B) A similar experiment using neuroblastoma cells overexpressing SorLA. (C) SorLAs inhibition of Ret phosphorylation was rescued by the presence of 10 μg/ml anti-SorLA antibodies during the experiment. (D) Inhibition of endogenous SorLA by the presence of excess propeptide (SorLApro) increases the survival of neuroblastoma cells whereas overexpression (SorLA) results in reduced survival. (E) Inhibition of SorLA by excess propeptide increases proliferation of neuroblastoma cells (F) Overexpression of SorLA inhibits GDNF-induced but not retinoic acid (RA)-induced neurite outgrowth.

FIG. 10. SorLA regulates GFRα1 and Ret subcellular localization throughout the brain

(A) SorLA is expressed throughout the central nervous system of young and old mice shown by Western blotting. (B) Immunohistochemistry showing the presence of SorLA in vesicles surrounding the soma of neurons throughout the cortex. (C) Altered subcellular localization of GFRα1 in SorLA−/− brain assessed by sucrose gradient centrifugation of brain homogenates from wild-type or SorLA−/− mice. (D) A similar experiment showing altered subcellular localization of Ret in SorLA−/− brain.

FIG. 11. SorLA is a general sorting receptor for GFRαs

(A) Cells stably expressing SorLA were transiently transfected with GFRα1-4 as indicated, which was subsequently immunoprecipitated using anti-GFRα1, -2, -3, and -4, respectively, followed by Western blotting using anti-SorLA. (B) Retrograde sorting of GFRα2 (green) in the presence of SorLA. (C) Altered subcellular localization of GFRα2 in SorLA−/− brains as assessed using sucrose gradient centrifugation.

FIG. 12. SorLA inhibition increases GDNF-induced survival of dopaminergic neurons

(A) Immunohistochemistry on the substantia nigra showing the presence of SorLA (green) in dopaminergic neurons (red). (B), Primary culture of dopaminergic neurons (red) grown on a glia cell layer. SorLA is expressed in both glia and neurons. (C) Colocalization of SorLA and GDNF in neurons and glia of primary dopaminergic neurons. (D) Colocalization of SorLA and GFRα1 in neurons and glia of primary dopaminergic neurons. (E) Survival of primary dopaminergic neurons requires GDNF and is promoted by anti-SorLA antibodies.

FIG. 13. SorLA−/− mice display hyperactivity and insensitivity to amphetamine

(A) SorLA−/− mice are hyperactive when tested in an open field. Hyperactivity is reversed by age. (B) Track plots of young and old SorLA−/− mice during 20 min in the open field. (C) SorLA−/− mice are insensitive to 10 mg/kg amphetamine. (D) Track plots of juvenile (less than 8 weeks old) wild-type and SorLA−/− mice during 40 min in the open field following injection of saline or amphetamine. (E) Track plots of adult (more than 12 weeks old) wild-type and SorLA−/− mice during 40 min in the open field following injection of saline or amphetamine.

FIG. 14. SorLA−/− mice show increased risk-taking behavior and attention deficits

(A) SorLA−/− mice are hyperactive when tested in an elevated plus maze. (B) SorLA−/− mice perform increased exits between arms in an elevated plus maze. (C) SorLA−/− mice perform increased entries into open arms relative to total exits between arms. (D) SorLA−/− mice spend an increased percentage of time in the open arms. (E) Falls of the elevated plus maze for wild-type (n=23) and SorLA−/− (n=12) mice. (F) Track plots of wild-type and SorLA−/− mice tested for 10 min in an elevated plus maze.

FIG. 15. Hypothetical model describing retrograde sorting of GDNF receptors by SorLA

FIG. 16. Truncation of GFRα1 domain 1 results in reduced affinity for SorLA.

(A) soluble full length GFRα1 (sGF Rα1 FL) binds to SorLA in a concentration-dependent manner with an estimated Kd=12 nM. (B) GFRα1 consists of three homologous domains. An N-terminal truncated soluble GFRα1 variant (s GFRα1 Δdomain 1) lacking a sequence stretch corresponding to domain 1 binds immobilized SorLA with a Kd=120 nM.

FIG. 17. The N-terminal 38 amino acids of mature GDNF bind to SorLA.

(A) fusion proteins between GST and the GDNF propeptide (GDNFpropep), the N-terminal 38 amino acids of mature GDNF (GDNFN-term), or the GDNF propeptide followed by the first 38 amino acids of mature GDNF (GDNFN-term propep) binding to immobilized SorLA. GST alone is included as negative control. (B) a peptide encompassing the first 38 amino acids of mature GDNF binds to SorLA in concentration-dependent manner.

FIG. 18. SorLA is a GDNF internalization receptor in the CNS

(A) Primary cultures of cortical glia cells express high levels of SorLA. (B) GDNF (10 nM, 15 min) is internalized by glia cells from wild-type but not Sorl1−/− mice.

FIG. 19. GDNF, not GFRα1, is sorted to lysosomes for degradation

(A) Inhibition of lysosomal degradation increases internalized GDNF levels in cells expressing SorLA and GFRα1. (B-C) Turnover of GFRα1 in the absence or presence of SorLA as assessed by metabolic labelling followed by pulse chase analysis.

FIG. 20. Increased GDNF levels in SorLA knockout mice

GDNF levels determined by ELISA in tissues from wild-type and Sorl1 knock-out mice. GDNF is increased in VTA and striatum of knockout animals (P=0.01, n=3; each comprising a pool of three animals).

FIG. 21. Inhibition of GDNF internalization and degradation by SorLA function blocking antibodies. (A) 293 cells expressing GFRa1 and SorLA were preincubated with non-specific rabbit IgG (10 ug/ml) for 2 h and subsequently with both GDNF (10 nM) and rabbit IgG for 15 min. Cells were then fixed and stained using GDNF antibodies and fluorescently labelled secondary antibodies. (B) a similar experiment using rabbit polyclonal antibodies raised against the extracellular domain of SorLA. The presence of anti-SorLA IgG increases surface GDNF immunofluorescence.

### DETAILED DESCRIPTION OF THE INVENTION

### Definitions

Adjuvant: Any substance whose admixture with an administered immunogenic determinant/antigen increases or otherwise modifies the immune response to said determinant.

Affinity: The interaction of a ligands with its binding site can be characterized in terms of a binding affinity. In general, high affinity ligand binding results from greater intermolecular force between the ligand and its receptor while low affinity ligand binding involves less intermolecular force between the ligand and its receptor. In general, high affinity binding involves a longer residence time for the ligand at its receptor binding site than is the case for low affinity binding. High affinity binding of ligands to receptors is often physiologically important when some of the binding energy can be used to cause a conformational change in the receptor, resulting in altered behavior of an associated ion channel or enzyme. A ligand that can bind to a receptor, alter the function of the receptor and trigger a physiological response is called an agonist for that receptor. Agonist binding to a receptor can be characterized both in terms of how much physiological response can be triggered and the concentration of the agonist that is required to produce the physiological response. High affinity ligand binding implies that a relatively low concentration of a ligand is adequate to maximally occupy a ligand binding site and trigger a physiological response. Low affinity binding implies that a relatively high concentration of a ligand is required before the binding site is maximally occupied and the maximum physiological response to the ligand is achieved. Ligand binding is often characterized in terms of the concentration of ligand at which half of the receptor binding sites are occupied, known as the dissociation constant (kd). Affinity is also the strength of binding between receptors and their ligands, for example between an antibody and its antigen.

Agonist: An agonist is a compound capable of increasing or effecting the activity of a receptor.

Antagonist: An antagonist is in this case synonymous with an inhibitor. An antagonist is a compound capable of decreasing the activity of an effector such as a receptor.

Specifically, an agonist or antagonist against SorLA or the GDNF-family ligand receptors (GFRα1-4) may be binding to their extracellular domain, e.g. the domain that mediates the specific binding between SorLA and the GDNF-family ligand receptors (e.g. as given in SEQ ID NO 5 or 7). Agents directed able to modulate the interaction between the SorLA and the GDNF-family ligand receptors may include soluble fragments of the SorLA or GDNF-family ligand receptors, antibodies directed to each of the recptors, natural binding partners such as GDNF or Neurotensin, or synthetic small organic compounds.

Antibody: The term “antibody” as referred to herein includes whole antibodies and any antigen binding fragment (i.e., “antigen-binding portion”) or single chain thereof.

“A whole antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region (abbreviated herein as C_{H}). Each light chain is comprised of a light chain variable region (abbreviated herein as VL) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody, as used herein, refers to one or more fragments of an antibody that retain the ability to specifically bind to an antigen. It has been shown that the antigen-binding function of an antibody can be performed by fragme is of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR),

and (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody.

A further example of an antigen binding-domain is immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. Such binding-domain immunoglobulin fusion proteins are further disclosed in US 2003/0118592 and US 2003/0133939 (both incorporated by reference in their entirety).

½

These antibody fragments are obtained using conventional techniques known to those with skill in the art, and the fragments are screened for utility in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “recombinant human antibody”, as used herein, includes all human antibodies

that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) also called affinity maturation and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “specific binding”′ refers to antibody binding to a predetermined antigen/epitope. Typically, the antibody binds with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when measured as apparent affinities based on IC₅₀ values in FACS, and binds to the predetermined antigen with an affinity corresponding to a K_{D} that is at least ten-fold lower, such as at least 100-fold lower than its affinity for binding to a non-specific antigen (e.g., BSA, casein) other than the predetermined antigen or a closely-related antigen.

Antibody Classes: Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (a), delta (d), epsilon (e), gamma (g) and mu (μ), respectively. The light chains of antibodies can be assigned to one of two clearly distinct types, called kappa (k) and lambda (l), based on the amino sequences of their constant domain. The subunit structures and three-dimensional configurations of different classes of immunoglobulins are well known.

Chimeric antibody: An antibody in which the variable regions are from one species of animal and the constant regions are from another species of animal. For example, a chimeric antibody can be an antibody having variable regions which derive from a mouse monoclonal antibody and constant regions which are human.

Complementarity determining region or CDR: Regions in the V-domains of an antibody that together form the antibody recognizing and binding domain.

Constant Region or constant domain or C-domain: Constant regions are those structural portions of an antibody molecule comprising amino acid residue sequences within a given isotype which may contain conservative substitutions therein. Exemplary heavy chain immunoglobulin constant regions are those portions of an immunoglobulin molecule known in the art as CH1, CH2, CH3, CH4 and CH5. An exemplary light chain immunoglobulin constant region is that portion of an immunoglobulin molecule known in the art as CL.

Human antibody framework: A molecule having an antigen binding site and essentially all remaining immunoglobulin-derived parts of the molecule derived from a human immunoglobulin.

Humanised antibody framework: A molecule having an antigen binding site derived from an immunoglobulin from a non-human species, whereas some or all of the remaining immunoglobulin-derived parts of the molecule is derived from a human immunoglobulin. The antigen binding site may comprise: either a complete variable domain from the non-human immunoglobulin fused onto one or more human constant domains; or one or more of the complementarity determining regions (CDRs) grafted onto appropriate human framework regions in the variable domain. In a humanized antibody, the CDRs can be from a mouse monoclonal antibody and the other regions of the antibody are human.

Immunoglobulin: The serum antibodies, including IgG, IgM, IgA, IgE and IgD.

Immunoglobulin isotypes: The names given to the Ig which have different H chains,

the names are IgG (IgG1,2,3,4), IgM, IgA (IgA1,2), sIgA, IgE, IgD.

Immunologically distinct: The phrase immunologically distinct refers to the ability to distinguish between two polypeptides on the ability of an antibody to specifically bind one of the polypeptides and not specifically bind the other polypeptide.

Monoclonal Antibody: The phrase monoclonal antibody in its various grammatical forms refers to a population of antibody molecules that contains only one species of antibody combining site capable of immunoreacting with a particular antigen. A monoclonal antibody thus typically displays a single binding affinity for any antigen with which it immunoreacts. A monoclonal antibody may contain an antibody molecule having a plurality of antibody combining sites, each immunospecific for a different antigen, e.g., a bispecific monoclonal antibody.

Polyclonal antibody: Polyclonal antibodies are a mixture of antibody molecules recognizing a specific given antigen, hence polyclonal antibodies may recognize different epitopes within said antigen.

Binding: The term “binding” or “associated with” refers to a condition of proximity between chemical entities or compounds, or portions thereof. The association may be non-covalent-wherein the juxtaposition is energetically favoured by hydrogen bonding or van der Waals or electrostatic interactions- or it may be covalent.

Binding site: The term “binding site” or “binding pocket”, as used herein, refers to a region of a molecule or molecular complex that, as a result of its shape, favourably associates with another molecule, molecular complex, chemical entity or compound. As used herein, the pocket comprises at least a deep cavity and, optionally a shallow cavity.

Fragments: The polypeptide fragments according to the present invention, including any functional equivalents thereof, may in one embodiment comprise less than 30 amino acid residues, for example less than 25 amino acid, less than 15 amino acids or less than 10 amino acids. Thus, it is contemplated that a fragment may e.g. comprise from about 2 to about 30 amino acids, or from about 2 to about 25, about 15 or about 10 amino acids, respectably.

GDNF-family ligand receptors is intended to include the receptors named GFRα1, 2, 3, and/or 4. The definition also includes isoforms thereof and, if specified, fragments or domains thereof as specified in e.g. SEQ ID NO 4 to 12.

SorLA is intended to include the SorLA receptor or fragments or domains thereof, as specified in e.g. SEQ ID NO 1 to 3.

Homology: The homology between amino acid sequences may be calculated using well known scoring matrices such as any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Ligand: a substance, compound or biomolecule such as a protein including receptors, that is able to bind to and form a complex with (a second) biomolecule to serve a biological purpose. In a narrower sense, it is a signal triggering molecule binding to a site on a target protein, by intermolecular forces such as ionic bonds, hydrogen bonds and Van der Waals forces. The docking (association) is usually reversible (dissociation). Actual irreversible covalent binding between a ligand and its target molecule is rare in biological systems. As opposed to the meaning in metalorganic and inorganic chemistry, it is irrelevant, whether or not the ligand actually binds at a metal site, as it is the case in hemoglobin. Ligand binding to receptors may alter the chemical conformation, i.e. the three dimensional shape of the receptor protein. The conformational state of a receptor protein determines the functional state of a receptor. The tendency or strength of binding is called affinity. Ligands include substrates, inhibitors, activators, non-self receptors, coreceptors and neurotransmitters. Radioligands are radioisotope labeled compounds and used in vivo as tracers in PET studies and for in vitro binding studies.

Pharmaceutical agent: The terms “pharmaceutical agent” or “drug” or “medicament” or “agent” refer to any therapeutic or prophylactic agent which may be used in the treatment (including the prevention, diagnosis, alleviation, or cure) of a malady, affliction, condition, disease or injury in a patient. Therapeutically useful genetic determinants, peptides, polypeptides and polynucleotides may be included within the meaning of the term pharmaceutical or drug. As defined herein, a “therapeutic agent”, “pharmaceutical agent” or “drug” or “medicament” or “agent” is a type of bioactive agent.

Pharmaceutical composition: or composition refers to any chemical or biological material, compound, or composition capable of inducing a desired therapeutic effect when properly administered to a patient.

Polypeptide: The term “polypeptide” as used herein refers to a molecule comprising at least two amino acids. The amino acids may be natural or synthetic.

The term “polypeptide” is also intended to include proteins, i.e. functional biomolecules comprising at least one polypeptide; when comprising at least two polypeptides, these may form complexes, be covalently linked or may be noncovalently.

Sequence identity: Sequence identity is determined in one embodiment by utilising fragments comprising at least 25 contiguous amino acids and having an amino acid sequence which is at least 80%, such as 85%, for example 90%, such as 95%, for example 99% identical to the amino acid sequence the protein in question, wherein the percent identity is determined with the algorithm GAP, BESTFIT, or FASTA in the Wisconsin Genetics Software Package Release 7.0, using default gap weights.

The invention provides evidence that SorLA—independently of GDNF—binds GFRα1 with a remarkably high affinity and that the two are efficiently coprecipitated from transfected cells (FIG. 6). When co-expressed in cells, SorLA conveys internalization and down-regulation of GFRα1, and mediates its retrograde transport to perinuclear (Golgi-)compartments thereby avoiding lysosomal degradation (FIG. 6). Notably, similar results with GFRα2, 3, and -4 suggest that SorLA may interact with all GFRα types, and that the functional implications described below may in fact concern not just GDNF but also neuturin, persephin and artemin. Moreover, expression of SorLA profoundly affects GDNF induction of Ret signalling, i.e. in Ret and GFRα1 positive cells responding to increasing concentrations of GDNF, overexpression of SorLA markedly inhibits Ret phosphorylation (FIG. 9). Studies of function performed on neuroblastoma cells and cultured neurons are in line with these results. Thus, the presence of SorLA hampers GDNF-induced survival, proliferation, and differentiation of neuroblastoma cells (FIG. 9), and the GDNF-induced survival of primary dopaminergic neurons is significantly enhanced when SorLA is functionally blocked by anti-SorLA antibodies (FIG. 12).

The inventors therefore propose that the interaction between SorLA and GDNF-family ligand receptors, such as GFRα1, are a key regulatory element in GDNF signalling, and that drugs (peptides, proteins, synthetic, small organic compounds) targeting the responsible binding sites in GDNF-family ligand receptors and/or SorLA can be used to promote or hamper GDNF functions by abrogating the GDNF-family ligand receptors binding to SorLA. The invention therefore relates to a method to hamper or reduce the survival, proliferation, and differentiation of neuroblastoma cells and, in another embodiment, increase the survival of neurons, such as e.g. dopaminergic neurons.

It is envisaged that various disease can be treated using these agents or drugs which are able to modulate the interaction between SorLA and GDNF-family ligand receptors and wherein the loss of neurons, such as e.g. dopaminergic neurons, is to be reversed or reduced and/or wherein the differentiation, proliferation and/or survival of neuroblastoma cells is to be reduced. These disease and injuries include neural cell death, spinal cord injury, peripheral nerve damage, cerebral ischemia, motor neuron disease, amyotrophic lateral sclerosis, chronic pain, neuropathic pain, epilepsy, cancer, Parkinson's disease, major depressive disorder, schizophrenia, attention deficit and hyperactivity disorder (ADHD), drug abuse, anxiety disorder, and/or bipolar disorder (manic depressive illness).

The inventors show that targeting of a GFRα:SorLA complex represents a completely new approach to the treatment of GFL-phenotypes in vivo—including behavioural phenotypes like ADHD and Parkinson's disease. Such strategy has a number of advantages over the above mentioned ongoing clinical and preclinical trials. For example, instead of delivering the ˜36 kDa GDNF family ligand dimer directly, the inventors propose the generation of a small molecule agonist or antagonist that crosses the blood-brain barrier and specifically modulates the interaction between SorLA and a GFRα receptor, thereby increasing the biological activity of endogenous GDNF family ligands.

Different agents can be envisaged to have these modulator effects on the SorLA and GDNF-family ligand receptor interaction. In particular, the agent used to modulate the interaction between the SorLA and GDNF-family ligand receptors is selected from proteins, peptides, antibodies or small organic compounds.

The agent may comprise the extracellular domain of SorLA (SEQ ID No 1), an isoform or a fragment thereof. By administering this polypeptide to a subject a competition between the GDNF-family ligand receptor found on the neuroblastoma cells or neurons, such as e.g. dopaminergic neurons, will happen whereby the interaction will be inhibited. Several fragments with affinity to the GDNF-family ligand receptor can be envisaged, in particular the N-terminal of SorLa of about 600 amino acids comprising the N-terminal Vps10p-domain of SorLA (SEQ ID NO 3). The inventors of the present invention additionally have shown that the the SorLA propeptide (SEQ ID NO 2) is effective for inhibiting this interaction.

Various modification of the extracellular domain of SorLA can be made without hampering the binding to the GDNF-family ligand receptors. It is thus envisaged that a polypeptide having a at least 80% sequence identity to SEQ ID NO 1, 2 or 3, such as at least 85%, 90%, 95% or 98% sequence identity to SEQ ID NO 1, 2 or 3 is able to have similar or related effects on the interactions.

The agents or drugs may of course also be selected from the extracellular domain of the GDNF-family ligand receptors, comprising the GFRα1-4 receptors, isoforms, or fragments thereof. In particular, the sequences as defined in SEQ ID 4, 5, 6, 7, 8, 9, 10, 11 or 12 or fragments thereof. SEQ ID NO 7 is the binding site to SorLA and a peptide comprising this sequence is in particular preferred.

Sequences having a at least 80% sequence identity to SEQ ID 4, 5, 6, 7, 8, 9, 10, 11 or 12, such as at least 85%, 90%, 95% or 98% sequence identity to SEQ ID 4, 5, 6, 7, 8, 9, 10, 11 or 12 are envisaged to have similar or related effects on the interactions.

It has also been shown that Neurotensin is also be able to inhibit this interaction. Thus according to one embodiment Neurotensin (SEQ ID 13) or a fragment thereof, such as defined in SEQ ID 14 or 15 may be used.

According to another embodiment of the invention the protein is GDNF which is a natural binding partner to the GDNF-family ligand receptor, GFRα1. Thus the invention also relates to GDNF, a fragment or an isoform thereof. The GDNF protein may comprise the sequence SEQ ID NO 16 or 18. According to another embodiment the protein is having at least 80% sequence identity to SEQ ID NO 16 or 18, such as at least 85%, 90%, 95% or 98% sequence identity to SEQ ID NO 16 or 18.

It has also been shown that the propeptide may be able to modulate the interaction between SorLA and GDNF-family ligand receptor, in particular GFRα1. Thus according to one embodiment the protein comprises the propeptide of GDNF, as defined in SEQ ID NO 17 or a protein having at least 80% sequence identity to SEQ ID NO 17, such as at least 85%, 90%, 95% or 98% sequence identity to SEQ ID NO 17.

The inventors further find that SorLA−/− transgenic mice exhibit a behavioural phenotype characterized by attention deficits and hyperactivity (FIG. 14), suggestive of altered dopaminergic activity—and an ADHD-like phenotype. Preliminary experiments using amphetamine-treatment of wt and transgenic mice support this hypothesis, inasmuch as SorLA−/− mice appear to be insensitive to amphetamine.

According to a particular embodiment the invention relates to a method to increase the extracellular levels of GDNF in the brain of a patient in the need thereof by modulating or inhibiting the interaction between SorLA and GFRα1, e.g. by inhibiting the internalisation and/or the degradation of GDNF is inhibited.

It is envisaged the this method can increases the survival of neurons, such as dopaminergic neurons by modulating the interaction between SorLA and GFRα1 and thus be suitable for treating diseases such as injury induced neural cell death, spinal cord injury, peripheral nerve damage, cerebral ischemia, motor neuron disease, amyotrophic lateral sclerosis, chronic pain, neuropathic pain, epilepsy, cancer, Parkinson's disease, major depressive disorder, schizophrenia, attention deficit and hyperactivity disorder (ADHD), drug abuse, anxiety disorder, and/or bipolar disorder (manic depressive illness).

An agent suitable for modulating or inhibiting the interaction between the SorLA and GFRα1 is an antibody directed against SorLA or GFRα1. This antibody preferably binds the extracellular domain of GFRα1 and/or SorLA comprising e.g. binding sites such as SEQ ID NO: 3, SEQ ID NO: 4, SEQ ID NO:5, SEQ ID NO: 6 or SEQ ID NO: 7.

The antibody may be polyclonal or monoclonal such as a humanized, chimeric, or single-chain antibody.

### Antibodies

Antibodies which bind to the same receptor targets as SorLA or the GDNF-family ligand receptors, such as GFRα1-4 of the invention can be prepared using an intact polypeptide or fragments containing small peptides of interest as the immunising antigen. The polypeptide used to immunise an animal may be obtained by recombinant DNA techniques or by chemical synthesis, and may optionally be conjugated to a carrier protein.

The preparation of polyclonal and monoclonal antibodies is well known in the art.

Polyclonal antibodies may in particular be obtained as described by, e.g., Green et al., “Production of Polyclonal Antisera” in Immunochemical 5 Protocols (Manson, Ed.); Humana Press, 1992, pages 1-5; by Coligan et al., “Production of Polyclonal Antisera in Rabbits, Rats, Mice and Hamsters” in Current Protocols in Immunology, 1992, Section 2.4.1, and by Ed Harlow and David Lane (Eds.) in “Antibodies; A laboratory manual” Cold Spring Harbor Lab. Press 1988. Monoclonal antibodies may in particular be obtained as described by, e.g., Kohler & Milstein, Nature, 1975, 256:495; Coligan et al., in Current Protocols in Immunology, 1992, Sections 2.5.1-2.6.7; and Harlow et al., in Antibodies: A Laboratory Manual; Cold Spring Harbor, Pub., 1988, page 726 (all incorporated by reference in their entirety).

Briefly, monoclonal antibodies may be obtained by injecting, e.g., mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce the antibodies to the antigen, and isolating the antibodies from the hybridoma cultures.

Monoclonal antibodies can be isolated and purified from hybridoma cultures by a variety of well-established techniques, including affinity chromatography with protein A Sepharose, size-exclusion chromatography, and ion-exchange chromatography, see. e.g. Coligan et al. in Current Protocols in Immunology, 1992, Sections 2.7.1-2.7.12, and Sections 2.9.1-2.9.3; and Barnes et al.: “Purification of Immunoglobulin 25 G (IgG)” in Methods in Molecular Biology; Humana Press, 1992, Vol. 10, Pages 79-104 (all incorporated by reference in their entirety). Polyclonal or monoclonal antibodies may optionally be further purified, e.g. by binding to and elution from a matrix to which the polypeptide, to which the antibodies were raised, is bound.

Antibodies against GFRα1-4 are commercially available for e.g. R&D Systems, such as Human GDNF MAb (Clone 27106), Mouse IgG1 Human GFR alpha-4 MAb (Clone 215725), Mouse IgG1 Human GFR alpha-2 MAb (Clone 129030), Mouse IgG2B Human GFR alpha-3 MAb (Clone 111004), Mouse IgG1 Human GFR alpha-1 MAb (Clone 260714), Mouse IgG1 Human/Rat GDNF Affinity Purified Polyclonal Ab, Goat IgG Human GFR alpha-4 Affinity Purified Polyclonal Ab, Goat IgG Human GFR alpha-1 Affinity Purified Polyclonal Ab, Goat IgG Human GFR alpha-2 Affinity Purified Polyclonal Ab, Goat IgG Human GFR alpha-3 Affinity Purified Polyclonal Ab, Goat IgG

Furthermore, the inventors have generated rabbit polyclonal antibodies raised against the entire extracellular domain of human SorLA. The SorLA extracellular domain was expressed in CHO cells and purified from culture supernatant using affinity chromatography.

These antibodies can function as a SorLA antagonist as demonstrated in FIGS. 9 and 12.

The inventors have in a similar manner generated a panel of mouse monoclonal antibodies raised against the entire extracellular domain of human SorLA. These can be selected based on their ability to function as a SorLA agonist or antagonist. The paratope of the selected antibody can then be cloned and a humanized antibody can be generated.

In one aspect the present invention relates to the use of an antibody capable of binding specifically to an epitope on the extracellular domain of GFRα1, 2, 3 or 4, in particular SEQ ID NO 4, 5, 6, 7, 8, 9, 10, 11 or 12 or sequences having a at least 80% sequence identity to SEQ ID 4, 5, 6, 7, 8, 9, 10, 11 or 12, such as at least 85%, 90%, 95% or 98% sequence identity to SEQ ID 4, 5, 6, 7, 8, 9, 10, 11 or 12 are envisaged to have similar or related effects on the interactions. SEQ ID NO 7 is the binding site to SorLA and a peptide comprising this sequence is in particular preferred.

According to another embodiment the invention relates to an antibody having an epitope on the extracellular domain of SorLA, in particular the sequences comprising the N-terminal Vpsp10p-domain (SEQ ID NO 3), SEQ ID No 1 or 2 or at least 80% sequence identity to SEQ ID NO 1, 2 or 3, such as at least 85%, 90%, 95% or 98% sequence identity to SEQ ID NO 1, 2 or 3 is able to have similar or related effects on the interactions.

In one embodiment, the antibody as defined herein above, is selected from the group consisting of: polyclonal antibodies, monoclonal antibodies, humanised antibodies, single chain antibodies, recombinant antibodies., chimeric antibodies or just an antigen portion thereof.

### Pharmaceutical Composition and Administration Forms

The main routes of drug delivery, in the treatment method are intravenous, oral, and topical. Other drug-administration methods, such as subcutaneous injection or via inhalation, which are effective to deliver the drug to a target site or to introduce the drug into the bloodstream, are also contemplated.

As the majority of compounds of the invention are proteins the most common way of administration is intravenous, intramuscular or subcutaneous administration, even though administration through intranasal application is also well described in the literature.

Appropriate dosage forms for such administration may be prepared by conventional techniques.

The compounds according to the invention may be administered as a single active agent or with at least one other active agent.

### Formulations

Whilst it is possible for the compounds of the present invention to be administered as the raw chemical, it is preferred to present them in the form of a pharmaceutical formulation.

Accordingly, the present invention further provides a pharmaceutical formulation, for medicinal application, which comprises the compound of the present invention and a pharmaceutically acceptable carrier or diluent.

In some embodiments of the invention is provided in a form of an antibody. A pharmaceutical formulation includes this antibody and an adjuvant.

Non-limiting examples of suitable adjuvants are selected from the group consisting of an immune targeting adjuvant, an immune modulating adjuvant such as a toxin a cytokine, and a mycobacterial derivative, an oil formulation, a polymer; a micelle forming adjuvant a saponin; an immunostimulating complex matrix (ISCOM matrix) a particle, DDA, aluminium adjuvants DNA adjuvants y-inulin, and an encapsulating adjuvant

The application of adjuvants include use of agents such as aluminum hydroxide or phosphate (alum)

According to the invention DDA (dimethyldioctadecylammonium bromide) is also a candidate for an adjuvant as is DNA and y-inulin, but also Freund's complete and incomplete adjuvants as well as quillaja saponins such as QuilA and QS21 are interesting as is RIBI Further possibilities are monophos-phoryl lipid A (MPL), the above mentioned C3 and C3d and mu-ramyl dipeptide (MDP)

Liposome formulations are also known to confer adjuvant effects and therefore liposome adjuvants are preferred according to the invention

Also immunostimulating complex matrix type (ISCOMO matrix) adjuvants are can be used according to the invention

Details relating to composition and use of immunostimulating complexes can eg. be found in Herein B et al. 1995, Clin Immunother 3. 461475 as well as Barr IG and Mitchell G F, 1996, Immunol and Cell Biol 74 8-25 (both incorporated by reference herein)

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents.

Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, 35 soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkylimidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophilelipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Transdermal Delivery

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987) (all incorporated by reference in their entirety). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

### Passive Transdermal Drug Delivery

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like (all incorporated by reference in their entirety). The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin.

Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical-considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, poly-vinylchlorides (PVC), polypropylenes, polycarbonates, poly-tetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%]. The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration.

The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler. When desired, formulations can be prepared with enteric coatings adapted for sustained or controlled release administration of the active ingredient. The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage 5 form can be a capsule, tablet, cachet, or lozenge itself, or it can be the appropriate number of any of these in packaged form.

In a the injection is intravenous, intramuscular, intraspinal, intraperitoneal, subcutaneous, a bolus or a continuous administration.

In one embodiment the pharmaceutical composition according to the present invention is administered at intervals of 30 minutes to 24 hours.

In a further embodiment the pharmaceutical composition according to the present invention is administered at intervals of 1 to 6 hours.

In a further embodiment the pharmaceutical composition according to the present invention is administered at intervals of 6 to 72 hours.

In another embodiment the pharmaceutical composition is administered at a dosage of between 10 μg to 500 mg per kg body mass.

The polypeptides and antibodies of the present invention may be administered in any manner, which is medically acceptable. This may include injections, by parenteral 10 routes such as intravenous, intravascular, intraarterial, subcutaneous, intramuscular, intratumor, intraperitoneal, intraventricular, intraepidural, intertracheat, intrathecal, intracerebroventricular, intercerebral, interpulmonary, or others as well as nasal, ophthalmic, rectal, or topical. Sustained release administration is also specifically included in the invention, by such means as depot injections or erodible implants.

Peroral administration is also conceivable provided the protein is protected against degradation in the stomach.

Administration may be by periodic injections of a bolus of the preparation, or may be made more continuous by intravenous or intraperitoneal administration from a reservoir which is external (e.g., an IV bag) or internal (e.g., a bioerodable implant, a bioartificial organ, a biocompatible capsule. See, e.g., U.S. Pat. Nos. 4,407,957, 5,798,113, and 5,800,828, each incorporated herein by reference. Intrapulmonary delivery methods and apparatus are described, for example, in U.S. Pat. Nos. 5,654,007, 5,780,014, and 5,814,607, each incorporated herein by reference. Apart from systemic delivery, delivery directly to the CNS behind the blood-brain or blood-retina barriers is also contemplated. Localised delivery may be by such means as delivery via a catheter to one or more arteries, such as the cerebral artery to the CNS. Methods for local pump-based delivery of protein formulations to the CNS are described in U.S. Pat. No. 6,042,579 (Medtronic) (incoporated by reference in its entirety).

The term “pharmaceutically acceptable carrier” means one or more organic or inorganic ingredients, natural or synthetic, with which the polypeptides or antibodies are combined to facilitate its application. A suitable carrier includes sterile saline although other aqueous and non-aqueous isotonic sterile solutions and sterile suspensions known to be pharmaceutically acceptable are known to those of ordinary skill in the art.

An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154 (all incoporated by reference in their entirety). In addition, it may be desirable to express the novel polypeptides of this invention, as well as other selected polypeptides, as lipoproteins, in order to enhance their binding to liposomes.

Various dosing regimes for systemic administration are contemplated. In one embodiment, methods of administering to a subject a formulation comprising the antibody or the polypeptides include administering said at a dosage of between 1 μg/kg to 30,000 μg/kg body weight of the subject, per dose.

In another embodiment, the dosage is between 10 μg/kg to 30,000 μg/kg body weight of th subject, per dose. In a further embodiment, the dosage is between 10 30 μg/kg to 10,000 μg/kg body weight of the subject, per dose. In a different embodiment, the dosage is between 5 μg/kg to 10,000 μg/kg body weight of the subject, per dose. In yet another embodiment, the dosage is between 25 μg/kg to 3,000 μg/kg body weight of the subject, per dose. In a most preferable embodiment, the dosage is between 50 μg/kg to 3,000 μg/kg body weight of the subject, per dose. Guidance as to particular dosages and methods of delivery is provided in the literature; see, for example, U.S. Pat. Nos. 4,657,760; 5,206,344; or 5,225,212 (all incoporated by reference in their entirety). It is anticipated that different formulations will be effective for different treatment compounds and different disorders, that administration targeting one organ or tissue, for example, may necessitate delivery in a manner different 5 from that to another organ or tissue.

### Assays

The present invention also encompass in vitro and/or in vivo assays for identifying new binding partners which are able to modulate the interaction between SorLA and GDNF-family ligand receptors, such as GFRα1, 2, 3 or 4, comprising both SorLA and GDNF-family ligand receptors. These assays may e.g. be cell based comprising cells expressing the receptors SorLA or GFRα1, 2, 3 or 4, or both, and measuring the response. Alternatively an in vitro assay may be performed whereby the new binding partners' ability to inhibit the interaction is measured. In the following is described various embodiments according to the present invention.

### Cell Based Assays

The invention also relates to various assays which can identify agents that are able to modulate the interaction between SorLa and the GDNF-family ligand receptor complex, in particular GFRα1.

Determination of binding, internalization or signaling by members of the Vps10p domain receptor family can be performed in cellular systems (Example 6). Cells expressing SorLA and GFRα1 and GDNF following e.g. transfection with plasmids encoding all three receptors, respectively, are incubated with a candidate agent (inhibitor/antagonist) compound. Said agent can e.g. represent an antibody against either one of the receptors, SorLA and GFRα1, binding ligands such as the SorLA propeptide or, fragments of the respective receptors. After incubation, the cells may be washed, the protein complexes crosslinked with e.g. dithiobis[succinimidylpropionate] (DSP, Pierce) and subsequently lysed. The thus obtained cell lysate may subsequently be incubated with antibody against either SorLA or GFRα1 or both bound to beads of e.g. sepharose. Precipitated complexes may then be eluted from the washed beads and analysed by Western blot.

Thus in one embodiment the invention relates to a cell based screening assay for identifying agents that can bind to the SorLa and GDNF-family ligand receptor complex comprising the steps of,
a) incubating the agent of interest with a cell expressing the SorLA receptor, GDNF and/or a GDNF-family ligand receptor
b) lysing the cells and incubating the cells with an antibody specific for SorLA or the GDNF-family ligand receptor, and
c) analyse the complex formation by western blot.

In another embodiment the invention relates to a cell based screening method for identifying agents capable of inhibiting SorLA

An antagonist directed against an entity of the SorLA:GFRα1:GDNF receptor complex may act as an inhibitor of the entire complex. Accordingly it is relevant to screen for agents capable of binding to e.g. the Vps10p-domain receptor entity.

Such a method is described in the example 8. Determination of binding, internalization or signalling by members of the Vps10pdomain receptor family may be performed in cellular systems. Cells expressing one of the receptors, either endogenously or following e.g. transfection with a plasmid containing the cDNA of the receptor, may be incubated with a radio-labeled ligand, in the absence and the presence respectively, of a candidate inhibitor/antagonist compound. After incubation, the cells may be washed to remove unspecific binding and subsequently harvested. The degree of binding of the candidate antagonist/inhibitor to the receptor is determined by using a conventional radioligand assay well known to those skilled in the art. See e.g. Bylund and Toews (1993) Am J Physiol. 265(5 Pt 1):L421-9 entitled “Radioligand binding methods: practical guide and tips”. Likewise, endocytosis/internalization may be determined as described in Nykjaer et al (1992) FEBS 300:13- and Nielsen et al (2001) EMBO J (both are incorporated by reference in their entirety).

Thus according to another embodiment the invention relates to a cell based screening method for identifying agents capable of inhibiting SorLA comprising the steps of,
a) incubating a cell expressing the SorLA receptor, GDNF and/or a GDNF-family ligand receptor with a radio-labelled agents,
b) washing the cells to remove unspecific binding,
c) harvesting the cells,
d) measuring the amount of binding

In a further embodiment a cell based screening method for identifying agents capable of modulating retrograde sorting of a GFRα receptor by SorLA is envisaged wherein surface localized GFRα receptor may be labeled either by a fluorescent tag or fluorescent antibodies in cells expressing both SorLA and GFRα receptor, and subsequently incubated with a SorLA agonist/antagonist for e.g. about 20 to about 30 minutes. The amount of internalized GFRα receptor can then subsequently be evaluated by using confocal microscopy as shown in FIG. 6 and FIG. 11.

Thus according to a further embodiment the invention relates a cell based screening method for identifying agents capable of modulating retrograde sorting of a GFRα receptor by SorLA comprising the steps of
a) labelling the agent by a fluorescent tag or fluorescent antibodies in cells expressing both the SorLA and GFRα receptor
b) subsequently incubated said cells with the SorLA agonist/antagonist for timeperiod
c) Analysing the amount of internalized GFRα receptor

In a still further embodiment a cell based screening method for identifying agents capable of increasing Ret phosphorylation is envisaged.

Neuroblastoma cells (e.g. SY5Y cells) may be stimulated by increasing concentrations of GDNF for defined periods of time in the absence or presence a SorLA antagonist or agonist. Cells may then be lysed and the cell lysates incubated with antibody against Ret (R&D Systems) coupled to Gammabind beads (GE Healthcare). Precipitated protein may then be eluted from the washed beads (acidic buffer and subsequent neutralization) and any phosphorylated Ret can be visualized by Western blotting using anti-phosphotyrosine (Milipore). (As shown in FIG. 9).

Thus the invention also relates to a cell based screening method for identifying agents capable of increasing Ret phosphorylation comprising the steps of
a) incubating neuriblastoma cells with GDNF at increasing concentrations at a defined amount of time in the presence or absence of a SorLA binding agent
b) lysing the cells,
c) immunoprecipitate the phosphorylated Ret using anti-phosphotyrosine antibodies, and
d) visualizing the phosphorylation

In a still further embodiment the invention relates to a cell based screening method for identifying agents capable of increasing cell survival

Neuroblastoma cells (e.g. SY5Y) may be harvested from a cell culture and resuspended in a growth medium such as DMEM without phenol red (LONZA) containing 1% glutamax and 1% P/S (penicillin and streptomycin). The cells may subsequently be plated in a 96 well. Next day, cells can then be stimulated by increasing concentrations of GDNF in the absence or presence of a SorLA antagonist or agonist and incubated for a certain time, e.g. for 72 hours, in their normal incubator. After incubation MultiTox-Fluor Multiplex Cytotoxicity Assay reagents (Promega) can be prepared and added to the cells as directed by supporting technical literature from manufacturer, and incubated. The fluorescence can therafter be measured with e.g. a Wallac VICTOR3™ 1420 Multilabel Counter (Perkin Elmer™ Lifesciences), where the signal is directly proportional to the survival.

Thus the invention also relates to a cell based screening method for identifying agents capable of increasing cell survival comprising the steps of,
a) incubating neuroblastoma cells increasing concentrations of GDNF in the absence or presence of a SorLA antagonist or agonist and incubated for a certain time
b) using a cytotoxic assay to determine the cell survival.

In another embodiment to test the survival is to grow the cells on coverslips instead of 96-well plate. After incubation the cells will then be fixed e.g. in 4% paraformaldehyde (PFA) in PBS for time period (such as 30 min at room temperature) and mounted onto slides. The cells can then be counted directly in a fluorescence microscope, as shown in FIG. 9D.

Thus the invention also relates to a cell based screening method for identifying agents capable of increasing cell survival comprising the steps of,
a) incubating neuroblastoma cells increasing concentrations of GDNF in the absence or presence of a SorLA antagonist or agonist and incubated for a certain time
b) fixing the cells
c) counting the cells.

### In Vitro Assays

An in vitro assay for identifying agents disrupting the interaction of GFRα1 and/or GDNF with SorLA

Determination of direct binding of an agent such as a small organic molecule, a peptide or a soluble receptor including but not limited to SorLA, GFRα1 and GDNF, to immobilized protein can be performed by e.g. surface plasmon resonance analysis (Biacore, Sweden) using e.g. CaHBS as standard running buffer (10 mM HEPES, pH 7.4, 140 mM NaCl, 2 mM CaCl2, 1 mM EGTA, and 0.005% Tween-20). Such an agent could be derived from the N-terminal of mature GDNF (FIG. 17) or the GFRα1 domain 1 (FIG. 16).

A biosensor chip from Biacore (CM5, cat. no. BR-1000-14) is activated using the NHS/EDC method as described by supplier followed by coating with SorLA. Several different approaches can be applied: Candidate agents can be identified by comparing the binding signal (response units) to a chip immobilized with one of the receptors and comparing this signal to an empty flow cell. In another approach, inhibition of an established agent can be monitored in the absence or presence of putative inhibitors. The difference in the signal depicts the inhibitory potential of the antagonist. The data collected can be analysed by fitting of sensorgrams for affinity estimations and inhibitory potential using the Biaevaluation version 3.1 program. The surface Plasmon resonance assay can easily be transform into other assays in which the Vps10p-domain receptor, the agent or the putative inhibitor is immobilized on a solid phase. For instance, receptors can be immobilized in e.g. Maxisorp microtiter wells from Nunc (cat. no. 439454) by incubation for certain time period (e.g. 16 hours at 4° C. in 50 mM NaHCO3, pH 9.6). After blocking using 5% bovine serum albumin (Sigma, cat. no. A9647) for 2 h at room temperature, the wells may be washed three times with MB buffer (10 mM HEPES, pH 7.4, 140 mM NaCl, 2 20 mM CaCl2, and 1 mM MgCl2) before incubation with a labelled ligand (e.g. iodinated) in the absence or presence of a various concentrations of a candidate inhibitor. Following incubation (e.g. overnight at 4° C.) and washing with MB buffer, bound radioactivity is released by adding 10% SDS. Nonspecific binding of tracer to wells coated only with bovine serum albumin can be determined and subtracted from the values determined in the binding experiments. The binding data point can be fitted to binding equations using the Prism software from GraphPad, version 4. Likewise, the antagonist can be labelled and binding to the immobilized receptor directly measured. In yet another setup, the receptor, ligand or antagonist can be immobilized on scintillation beads and binding measured in a scintillation proximity assay in which the receptor-binding molecule has been labelled using radioactivity.

Thus the present invention also relates to an in vitro assay for identifying agents disrupting the interaction of GFRα1 and/or GDNF with SorLA comprising the steps of
a) immobilizing GFRα1 and/or SorLA in a biosensor chip
b) applying the agent, and
c) comparing this signal to standard

### OR

a) immobilizing the agent, GFRα1, GDNF or SorLA on a solid phase,
b) incubating these with a labelled counterpart (e.g. iodinated agent, GFRα1, GDNF and/or SorLA) in the absence or presence of a various concentrations said agent, and
c) counting or measuring the binding.

### FURTHER EMBODIMENTS

1. A method to increase the survival of neurons, such as dopaminergic neurons, by modulating the interaction between SorLA and GDNF-family ligand receptors.
2. The method according to embodiment 2, wherein the GDNF-family ligand receptors are selected from the group comprising GFRα1, 2, 3, and/or 4.
3. The method according to embodiment 1 or 2, wherein the agent used to modulate the interaction between the SorLA and GDNF-family ligand receptors is selected from proteins, peptides, antibodies or small organic compounds.
4. The method according to embodiment 3, wherein the agent is the extracellular domain of SorLA (SEQ ID NO 1), an isoform or a fragment thereof.
5. The method according to embodiment 4, wherein the fragment comprises or is a fragment from the N-terminal Vps10p-domain of SorLA (SEQ ID NO 3).
6. The method according to embodiment 3, wherein the agent is the SorLA propeptide (SEQ ID NO 2) or a fragment thereof.
7. The method according to any one of embodiments 2 to 5, having a at least 80% sequence identity to SEQ ID NO 1, 2 or 3, such as at least 85%, 90%, 95% or 98% sequence identity to SEQ ID NO 1, 2 or the 3.
8. The method according to embodiment 3, wherein the agent is selected from the extracellular domain of the GFRα1-4 receptors, isoforms, or fragments thereof.
9. The method according to embodiment 8, wherein the GFRα receptors comprises the sequences as defined in SEQ ID 4, 5, 6, 7, 8, 9, 10, 11 or 12 or fragments thereof.
10. The method according to embodiment 8, having a at least 80% sequence identity to SEQ ID 4, 5, 6, 7, 7, 8, 9, 10, 11 or 12, such as at least 85%, 90%, 95% or 98% sequence identity to SEQ ID 4, 5, 6, 7, 8, 9, 10, 11 or 12.
11. The method according to embodiment 3, wherein the protein comprises or consist of Neurotensin (SEQ ID 13) or a fragment thereof, such as defined in SEQ ID 14 or 15.
12. The method according to embodiment 3, wherein the protein is GDNF, a fragment or an isoform thereof.
13. The method according to embodiment 12, wherein the GDNF comprises or consist of the sequence SEQ ID NO 16 or 18.
14. The method according to embodiment 13, having at least 80% sequence identity to SEQ ID NO 16 or 18, such as at least 85%, 90%, 95% or 98% sequence identity to SEQ ID NO 16 or 18.
15. The method according to embodiment 3, wherein the protein comprises or consist of the propeptide of GDNF, as defined in SEQ ID NO 17.
16. The method according to embodiment 15, having at least 80% sequence identity to SEQ ID NO 17, such as at least 85%, 90%, 95% or 98% sequence identity to SEQ ID NO 17.
17. The method according to embodiment 3, wherein the antibody has an epitope on the extracellular domain of GFRα1, 2, 3 or 4.
18. The antibody according to embodiment 17, being either monoclonal or polyclonal and having an epitope within any of the sequences as defined in any one of embodiments 8, 9 or 10.
19. The method according to embodiment 3, wherein the antibody has an epitope on the extracellular domain of SorLA.
20. The method according to embodiment 19, being either monoclonal or polyclonal and having an epitope within any of the sequences as defined in any one of embodiments 4, 5, 6 or 7.
21. A pharmaceutical composition comprising an agent as defined in any one of embodiments 1-16 in combination with one or more pharmaceutically acceptable carriers or diluents.
22. A pharmaceutical composition comprising an antibody as defined in any one of embodiments 17-20 and an adjuvant.
23. Use of an agent as defined in any one of embodiments 1 to 20 or a pharmaceutical composition according to any one of embodiments 21 and 22, to increase the survival of neurons, such as dopaminergic neurons.
24. An agent as defined in embodiments 1-20 or a pharmaceutical composition according to any one of embodiments 21 and 22 for use in the treatment of a disease associated with the loss of neurons, such as dopaminergic neurons, and/or wherein the survival of neurons, such as dopaminergic neurons, are desired.
25. The use according to embodiment 24, wherein the diseases are selected from the group comprising injury induced neural cell death, spinal cord injury, peripheral nerve damage, cerebral ischemia, motor neuron disease, amyotrophic lateral sclerosis, chronic pain, neuropathic pain, epilepsy, cancer, Parkinson's disease, major depressive disorder, schizophrenia, attention deficit and hyperactivity disorder (ADHD), drug abuse, anxiety disorder, and/or bipolar disorder (manic depressive illness).
26. A method for treating a disease associated with the loss of neurons, such as dopaminergic neurons, and/or wherein the survival of neurons, such as dopaminergic neurons, are desired by administering an effective amount of an agent as defined in any one of embodiments 1-20 or a pharmaceutical composition according to any one of embodiments 21 or 22.
27. A method according to embodiment 26 for treating a disease according to embodiment 25.
28. The use of a compound according to any of the embodiments 1 to 20 or a pharmaceutical composition according to any one of embodiments 21 and 22 for the preparation of a medicament for the treatment of a disease responsive according to any one of embodiments 24 or 25.
29. An in vitro or in vivo assay for identifying a binding partner which is able to modulate the interaction between SorLA and GDNF-family ligand receptors, such as GFRα1, 2, 3 or 4, comprising the SorLA and/or the GDNF-family ligand receptor.

### REFERENCES

Andersen, O. M., J. Reiche, et al. (2005). “Neuronal sorting protein-related receptor sorLA/LR11 regulates processing of the amyloid precursor protein.” Proc Natl Acad Sci USA 102(38): 13461-6.
Beck, K. D., J. Valverde, et al. (1995). “Mesencephalic dopaminergic neurons protected by GDNF from axotomy-induced degeneration in the adult brain.” Nature 373(6512): 339-41.
Cacalano G, Farinas I, Wang L C, Hagler K, Forgie A, Moore M, Armanini M, Phillips H, Ryan A M, Reichardt L F, Hynes M, Davies A, Rosenthal A (1998) GFRalphal is an essential receptor component for GDNF in the developing nervous system and kidney. Neuron 21(1): 53-62
Chauhan N B, Siegel G J, Lee J M (2001) Depletion of glial cell line-derived neurotrophic factor in substantia nigra neurons of Parkinson's disease brain. J Chem Neuroanat 21(4): 277-288
Ciechanowski, P. S., W. J. Katon, et al. (2000). “Depression and diabetes: impact of depressive symptoms on adherence, function, and costs.” Arch Intern Med 160(21): 3278-85.
Davie, C. A. (2008). “A review of Parkinson's disease.” Br Med Bull 86: 109-27.
Dodson, S. E., M. Gearing, et al. (2006). “LR11/SorLA expression is reduced in sporadic Alzheimer disease but not in familial Alzheimer disease.” J Neuropathol Exp Neurol 65(9): 866-72.
Durbec P L, Larsson-Blomberg L B, Schuchardt A, Costantini F, Pachnis V (1996) Common origin and developmental dependence on c-ret of subsets of enteric and sympathetic neuroblasts. Development 122(1): 349-358
Enomoto H, Araki T, Jackman A, Heuckeroth R O, Snider W D, Johnson E M, Jr., Milbrandt J (1998) GFR alpha1-deficient mice have deficits in the enteric nervous system and kidneys. Neuron 21(2): 317-324
Gash, D. M., G. A. Gerhardt, et al. (1998). “Effects of glial cell line-derived neurotrophic factor on the nigrostriatal dopamine system in rodents and non-human primates.” Adv Pharmacol 42: 911-5.
Gill, S. S., N. K. Patel, et al. (2003). “Direct brain infusion of glial cell line-derived neurotrophic factor in Parkinson disease.” Nat Med 9(5): 589-95.
Golden J P, Baloh R H, Kotzbauer P T, Lampe P A, Osborne P A, Milbrandt J, Johnson E M, Jr. (1998) Expression of neurturin, GDNF, and their receptors in the adult mouse CNS. J Comp Neurol 398(1): 139-150
Granholm A C, Reyland M, Albeck D, Sanders L, Gerhardt G, Hoernig G, Shen L, Westphal H, Hoffer B (2000) Glial cell line-derived neurotrophic factor is essential for postnatal survival of midbrain dopamine neurons. J Neurosci 20(9): 3182-3190
Jacobsen L, Madsen P, Nielsen M S, Geraerts W P, Gliemann J, Smit A B, Petersen C M (2002) The sorLA cytoplasmic domain interacts with GGA1 and -2 and defines minimum requirements for GGA binding. FEBS Lett 511(1-3): 155-158
Kanaki, T., H. Bujo, et al. (1998). “Developmental regulation of LR11 expression in murine brain.” DNA Cell Biol 17(8): 647-57.
Kearns, C. M. and D. M. Gash (1995). “GDNF protects nigral dopamine neurons against 6-hydroxydopamine in vivo.” Brain Res 672(1-2): 104-11.
Kramer E R, Aron L, Ramakers G M, Seitz S, Zhuang X, Beyer K, Smidt M P, Klein R (2007) Absence of Ret signaling in mice causes progressive and late degeneration of the nigrostriatal system. PLoS Biol 5(3): e39
Kupfer, D. J. (2005). “The increasing medical burden in bipolar disorder.” Jama 293(20): 2528-30.
Lang, A. E., S. Gill, et al. (2006). “Randomized controlled trial of intraputamenal glial cell line-derived neurotrophic factor infusion in Parkinson disease.” Ann Neurol 59(3): 459-66.
Lin, L. F. (1993) et al. GDNF: a glial cell-derived neurotrophic factor for midbrain dopaminergic neurons. Science May 21: 1130-2
Manji, H. K., W. C. Drevets, et al. (2001). “The cellular neurobiology of depression.” Nat Med 7(5): 541-7.
Marcusson, E. G., B. F. Horazdovsky, et al. (1994). “The sorting receptor for yeast vacuolar carboxypeptidase Y is encoded by the VPS10 gene.” Cell 77(4): 579-86.
Mazella, J., N. Zsurger, et al. (1998). “The 100-kDa neurotensin receptor is gp95/sortilin, a non-G-protein-coupled receptor.” J Biol Chem 273(41): 26273-6
Michelson, D., C. Stratakis, et al. (1996). “Bone mineral density in women with depression.” N Engl J Med 335(16): 1176-81.
Moore M W, Klein R D, Farinas I, Sauer H, Armanini M, Phillips H, Reichardt L F, Ryan A M, Carver-Moore K, Rosenthal A (1996) Renal and neuronal abnormalities in mice lacking GDNF. Nature 382(6586): 76-79
Motoi, Y., T. Aizawa, et al. (1999). “Neuronal localization of a novel mosaic apolipoprotein E receptor, LR11, in rat and human brain.” Brain Res 833(2): 209-15.
Munck Petersen, C., M. S. Nielsen, et al. (1999). “Propeptide cleavage conditions sortilin/neurotensin receptor-3 for ligand binding.” Embo J 18(3): 595-604.
Musselman, D. L., D. L. Evans, et al. (1998). “The relationship of depression to cardiovascular disease: epidemiology, biology, and treatment.” Arch Gen Psychiatry 55(7): 580-92.
Nishino J, Mochida K, Ohfuji Y, Shimazaki T, Meno C, Ohishi S, Matsuda Y, Fujii H, Saijoh Y, Hamada H (1999) GFR alpha3, a component of the artemin receptor, is required for migration and survival of the superior cervical ganglion. Neuron 23(4): 725-736
Nutt, J. G., K. J. Burchiel, et al. (2003). “Randomized, double-blind trial of glial cell line-derived neurotrophic factor (GDNF) in P D.” Neurology 60(1): 69-73.
Nykjaer, A., R. Lee, et al. (2004). “Sortilin is essential for proNGF-induced neuronal cell death.” Nature 427(6977): 843-8.
Offe, K., S. E. Dodson, et al. (2006). “The lipoprotein receptor LR11 regulates amyloid beta production and amyloid precursor protein traffic in endosomal compartments.” J Neurosci 26(5): 1596-603.
Rogaeva, E., Y. Meng, et al. (2007). “The neuronal sortilin-related receptor SORL1 is genetically associated with Alzheimer disease.” Nat Genet 39(2): 168-77.
Rossi J, Luukko K, Poteryaev D, Laurikainen A, Sun Y F, Laakso T, Eerikainen S, Tuominen R, Lakso M, Rauvala H, Arumae U, Pasternack M, Saarma M, Airaksinen M S (1999) Retarded growth and deficits in the enteric and parasympathetic nervous system in mice lacking GFR alpha2, a functional neurturin receptor. Neuron 22(2): 243-252
Sager, K. L., J. Wuu, et al. (2007). “Neuronal LR11/sorLA expression is reduced in mild cognitive impairment.” Ann Neurol 62(6): 640-7
Sauer, H., C. Rosenblad, et al. (1995). “Glial cell line-derived neurotrophic factor but not transforming growth factor beta 3 prevents delayed degeneration of nigral dopaminergic neurons following striatal 6-hydroxydopamine lesion.” Proc Natl Acad Sci USA 92(19): 8935-9.
Scherzer, C. R., K. Offe, et al. (2004). “Loss of apolipoprotein E receptor LR11 in Alzheimer disease.” Arch Neurol 61(8): 1200-5.
Schulz, R., S. R. Beach, et al. (2000). “Association between depression and mortality in older adults: the Cardiovascular Health Study.” Arch Intern Med 160(12): 1761-8.
Sian J, Gerlach M, Youdim M B, Riederer P (1999) Parkinson's disease: a major hypokinetic basal ganglia disorder. J Neural Transm 106(5-6): 443-476
Souza et al. (2010). J Psychiatr Res.
Spoelgen, R., C. A. von Arnim, et al. (2006). “Interaction of the cytosolic domains of sorLA/LR11 with the amyloid precursor protein (APP) and beta-secretase betasite APP-cleaving enzyme.” J Neurosci 26(2): 418-28.
Syed Z, Dudbridge F, Kent L (2007) An investigation of the neurotrophic factor genes GDNF, NGF, and NT3 in susceptibility to ADHD. Am J Med Genet B Neuropsychiatr Genet 144B(3): 375-378
Teng, H. K., K. K. Teng, et al. (2005). “ProBDNF induces neuronal apoptosis via activation of a receptor complex of p75NTR and sortilin.” J Neurosci 25(22): 5455-63
Tomac A C, Grinberg A, Huang S P, Nosrat C, Wang Y, Borlongan C, Lin S Z, Chiang Y H, Olson L, Westphal H, Hoffer B J (2000) Glial cell line-derived neurotrophic factor receptor alpha1 availability regulates glial cell line-derived neurotrophic factor signaling: evidence from mice carrying one or two mutated alleles. Neuroscience 95(4): 1011-1023
Tomac, A., E. Lindqvist, et al. (1995). “Protection and repair of the nigrostriatal dopaminergic system by GDNF in vivo.” Nature 373(6512): 335-9.
Westergaard, U. B., E. S. Sorensen, et al. (2004). “Functional organization of the sortilin Vps10p domain.” J Biol Chem 279(48): 50221-9.
Williams et al. (2007). Schizophr Res
Zaman V, Boger H A, Granholm A C, Rohrer B, Moore A, Buhusi M, Gerhardt G A, Hoffer B J, Middaugh L D (2008) The nigrostriatal dopamine system of aging GFRalpha-1 heterozygous mice: neurochemistry, morphology and behavior. Eur J Neurosci 28(8): 1557-1568
Zurn A D, Widmer H R, Aebischer P (2001) Sustained delivery of GDNF: towards a treatment for Parkinson's disease. Brain Res Brain Res Rev 36(2-3): 222-229.

### EXAMPLES

### Example 1

### Demonstration of a SorLA:GFRα1 complex

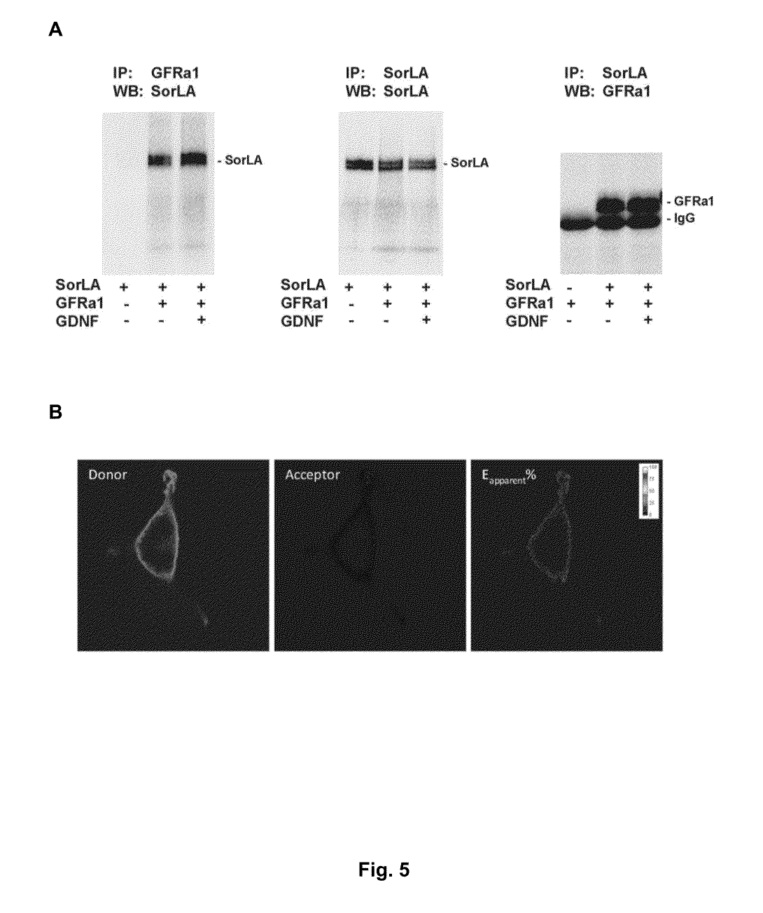

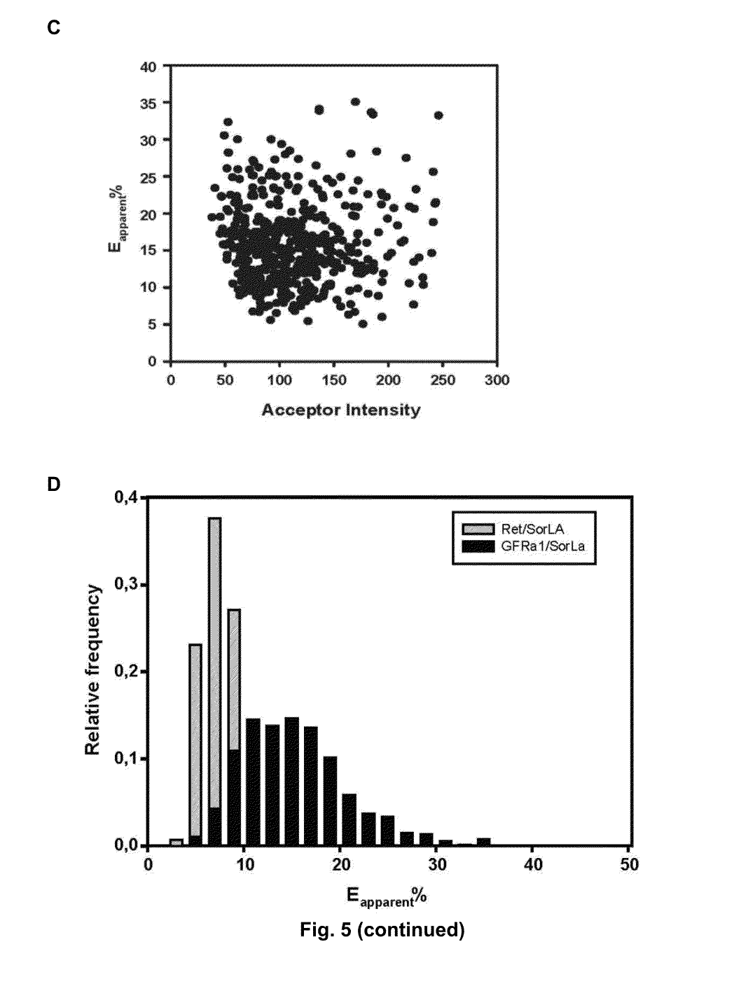

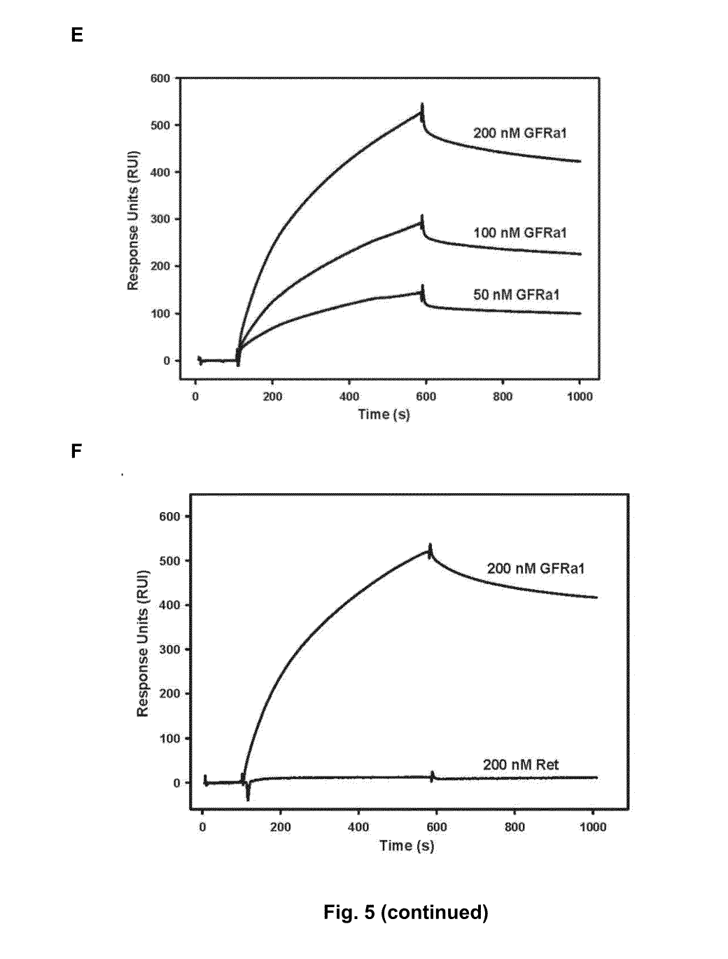

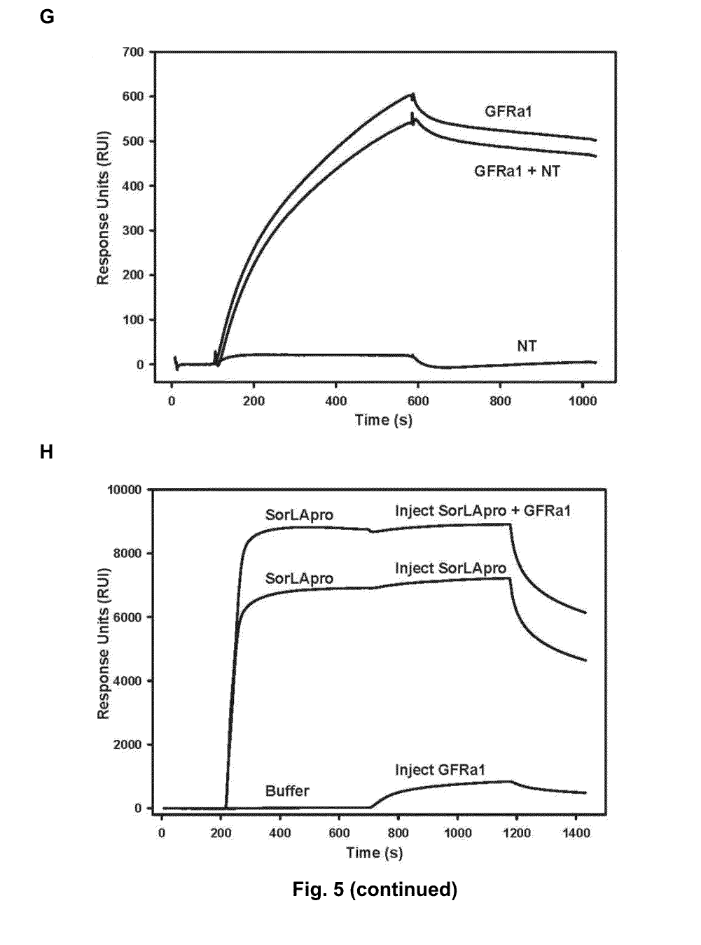

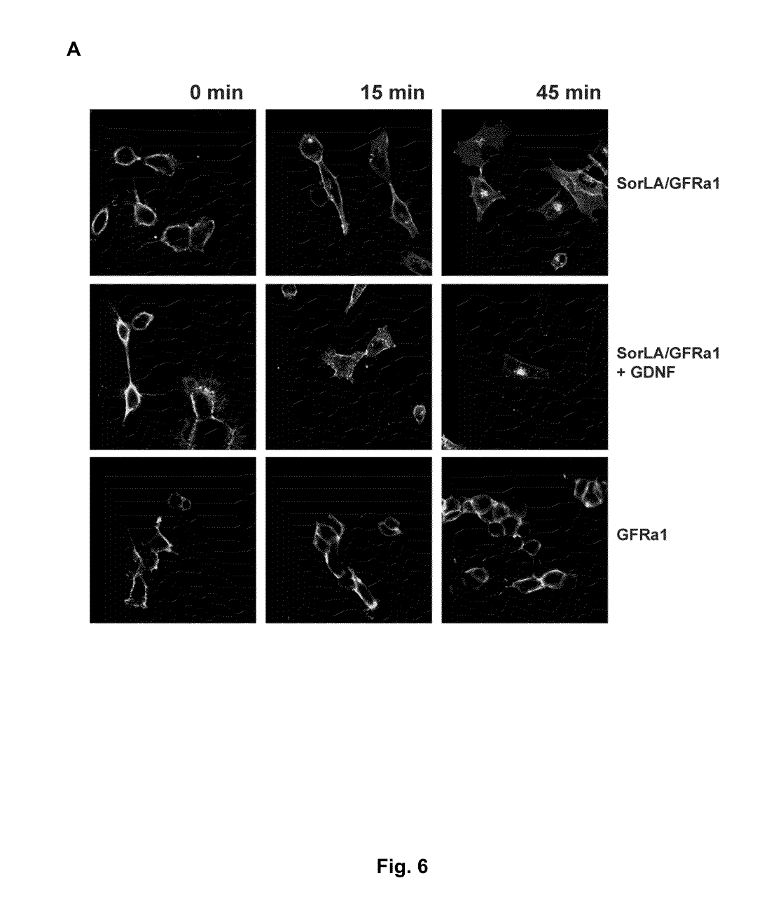

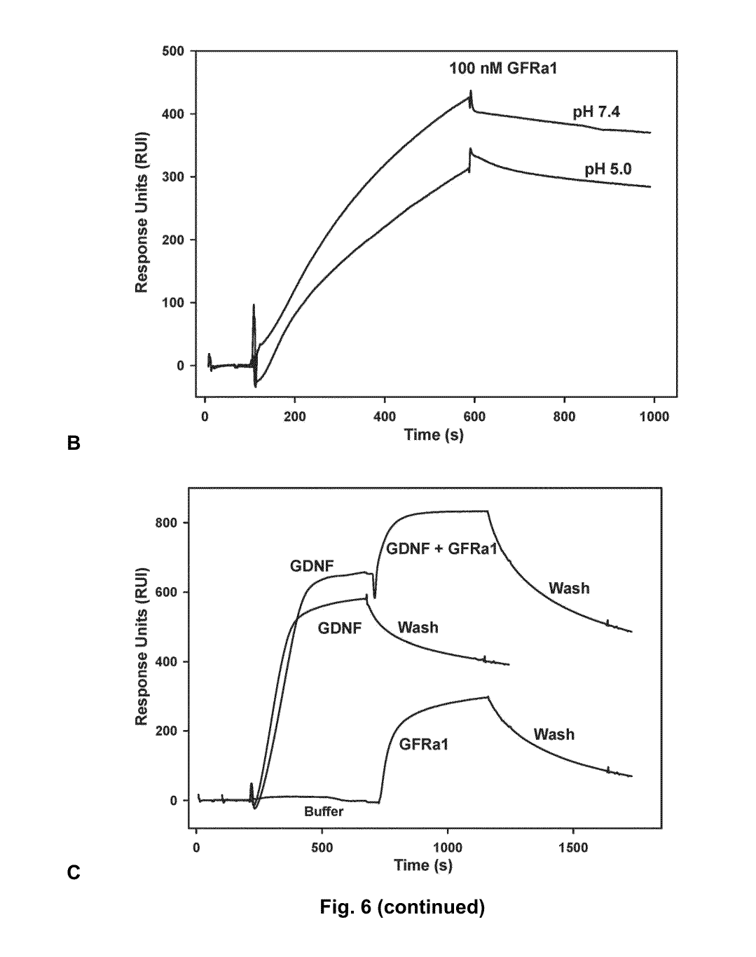

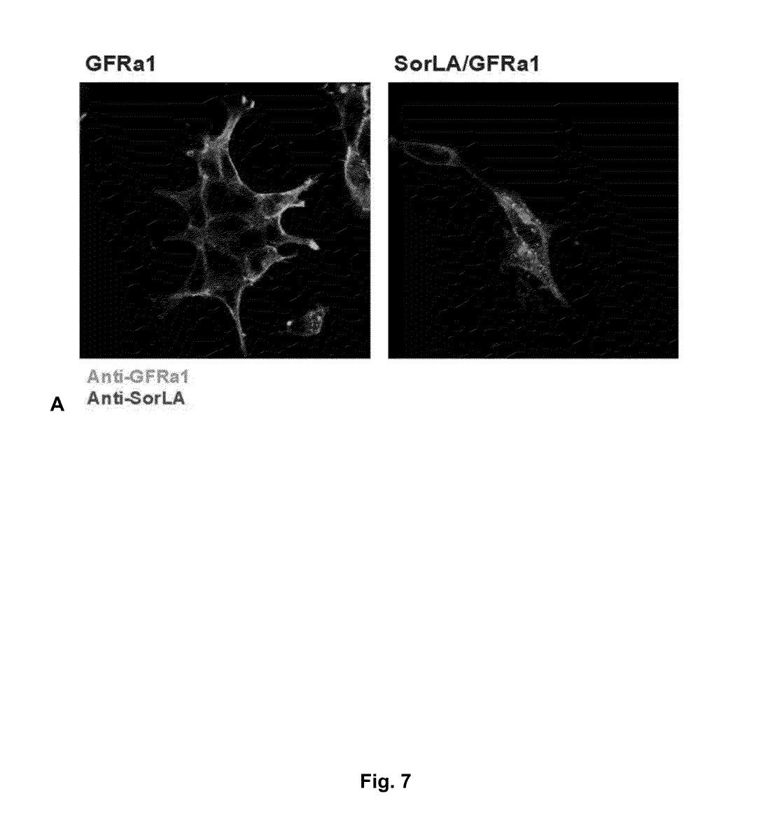

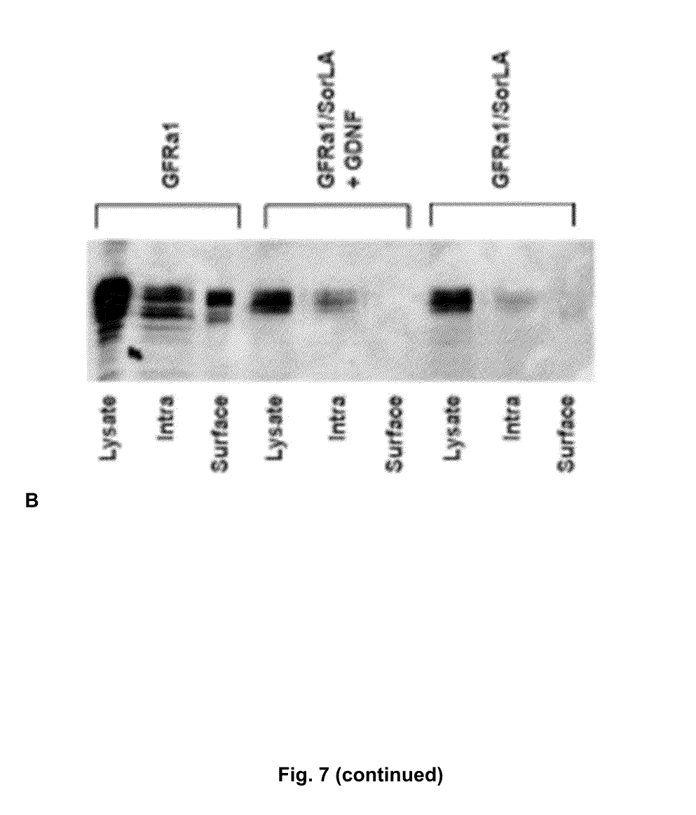

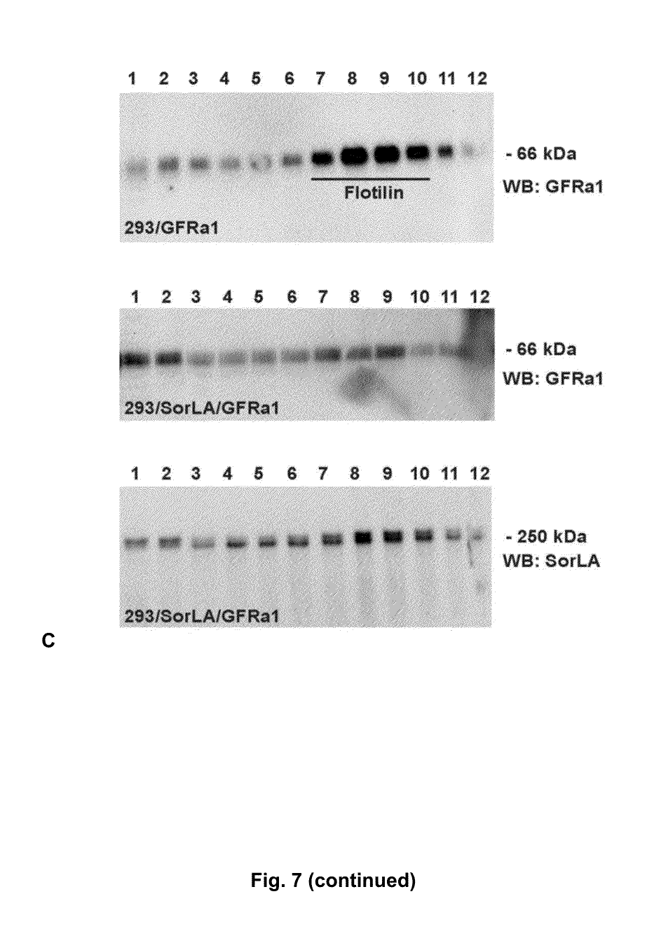

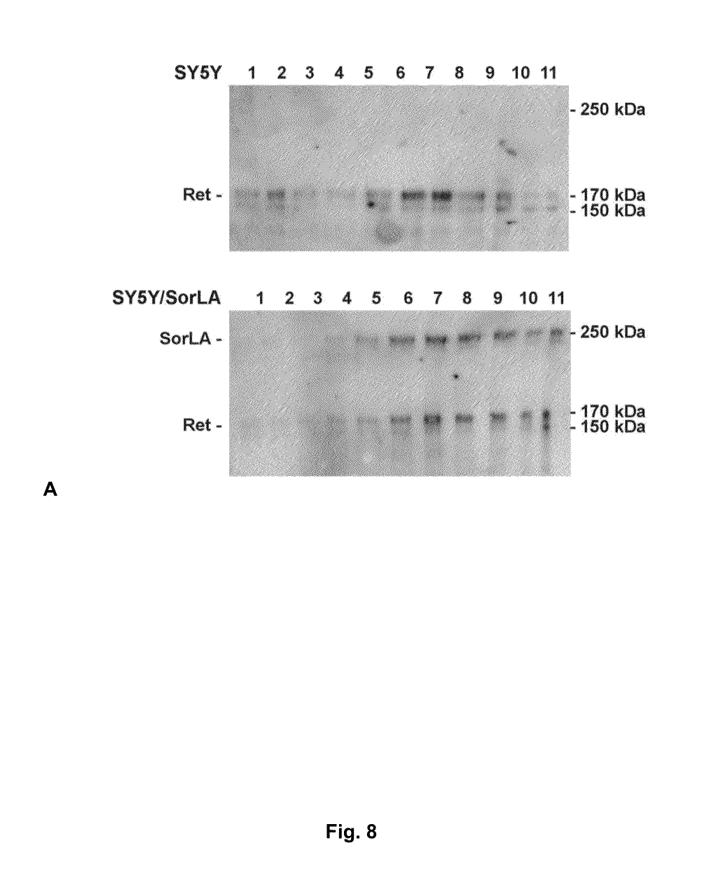

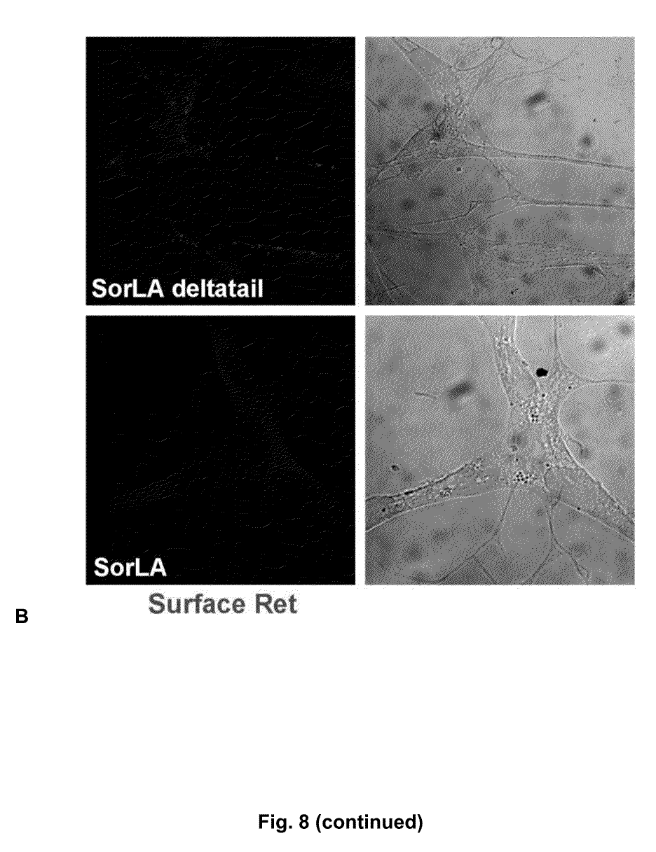

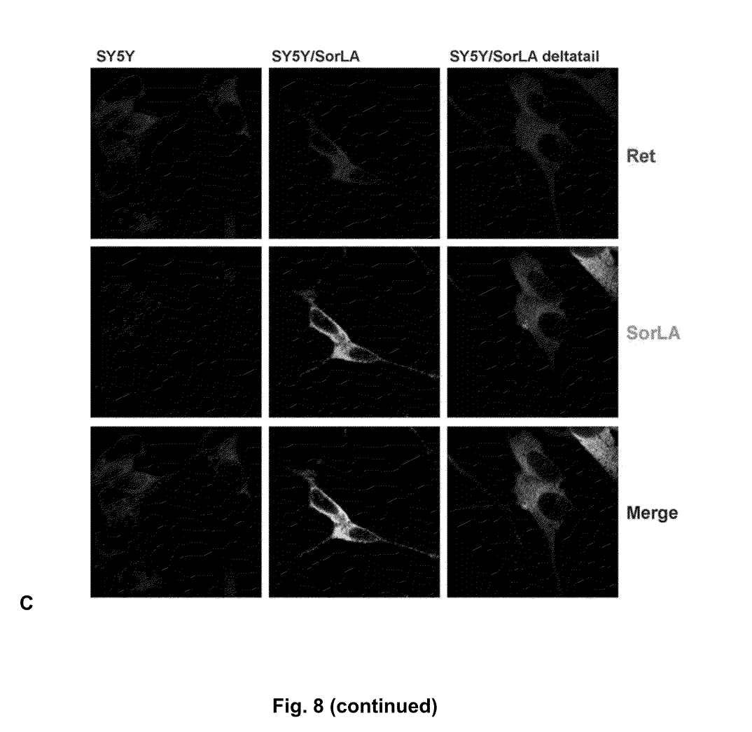

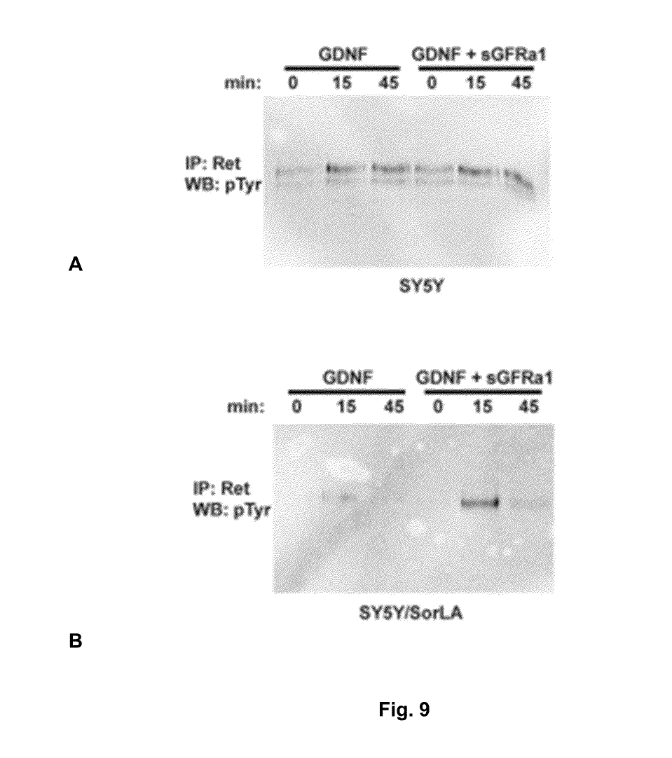

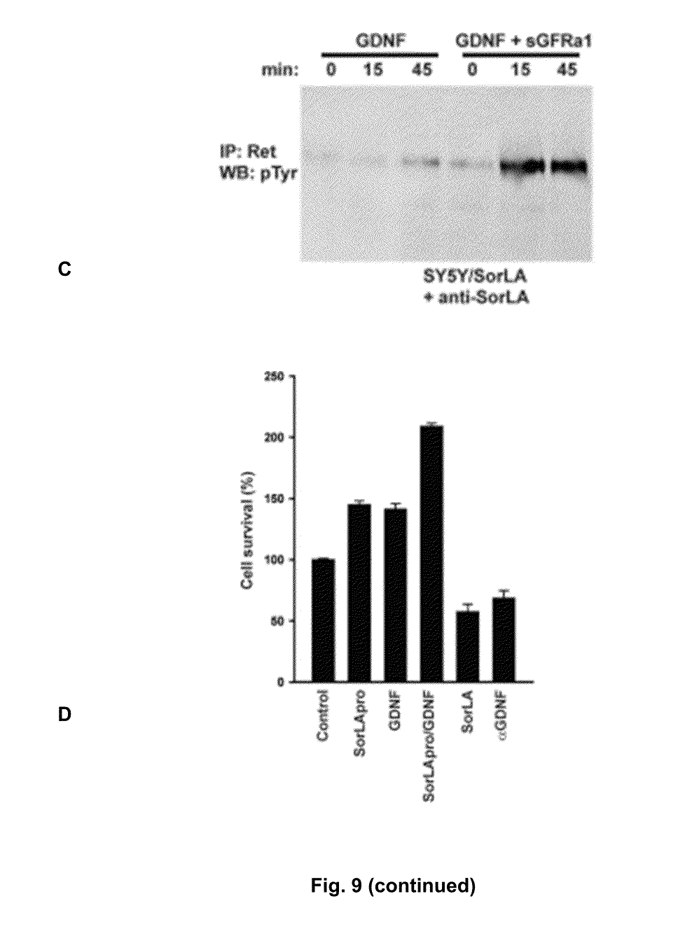

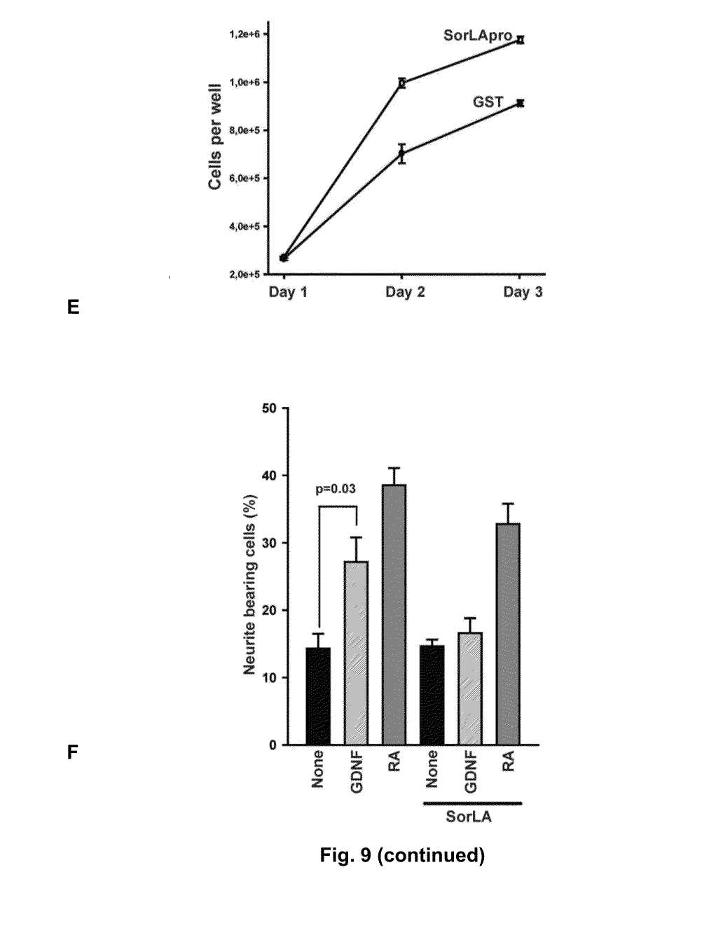

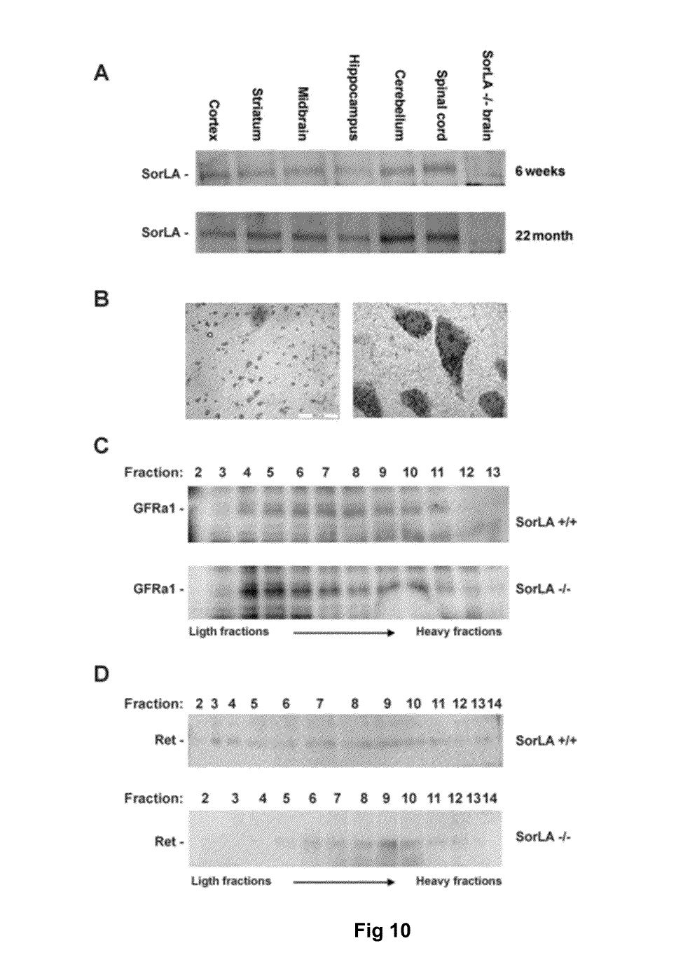

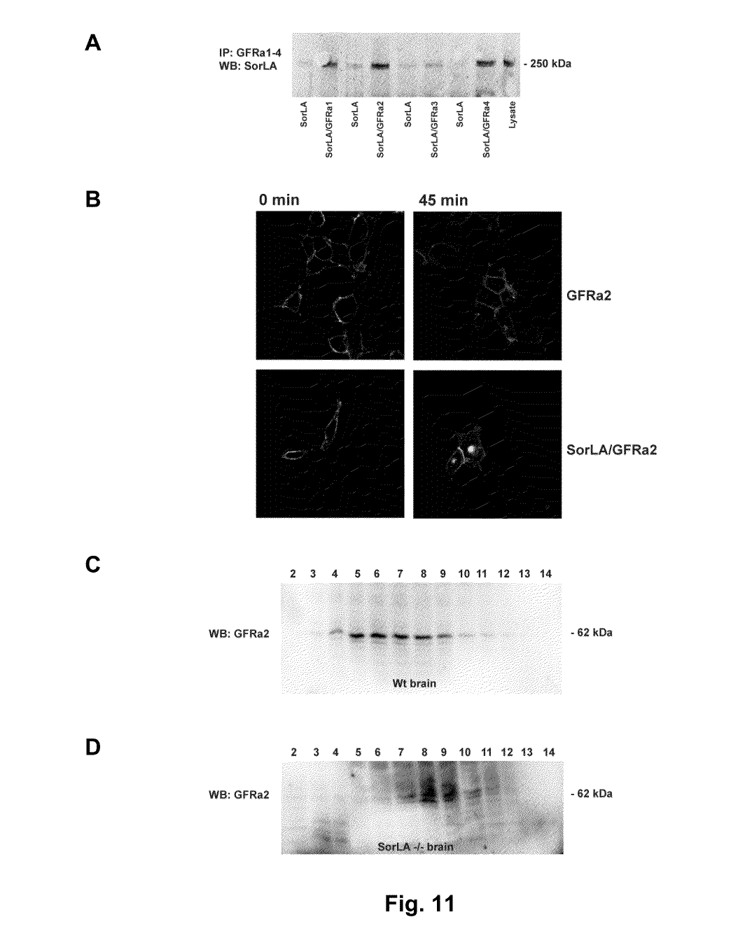

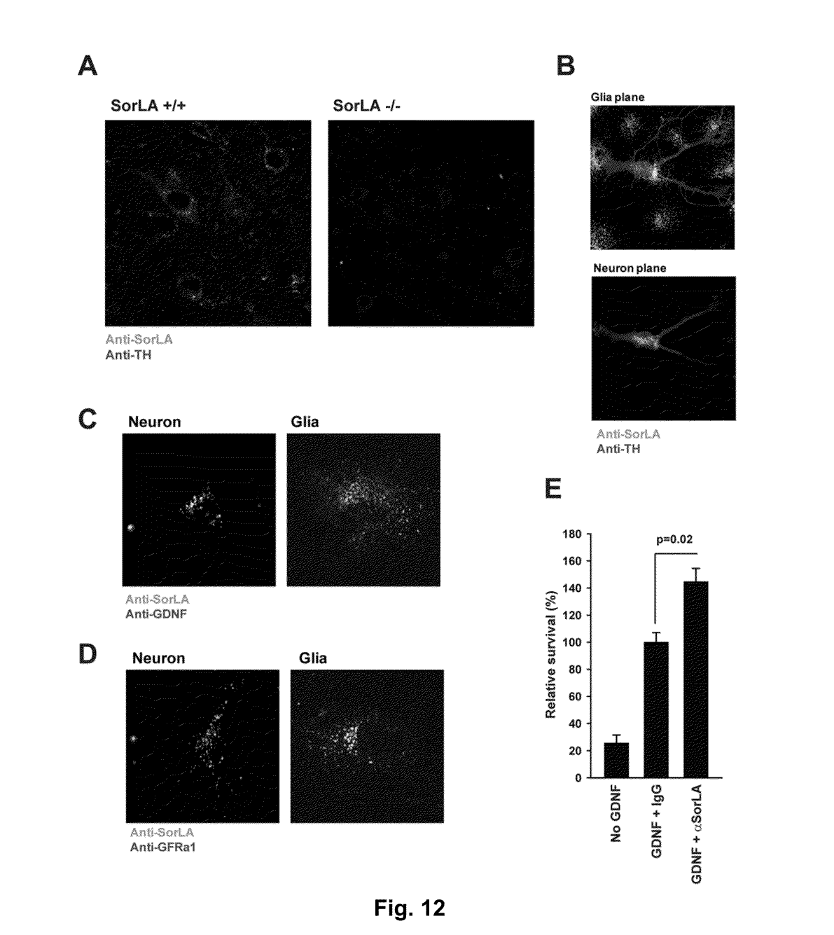

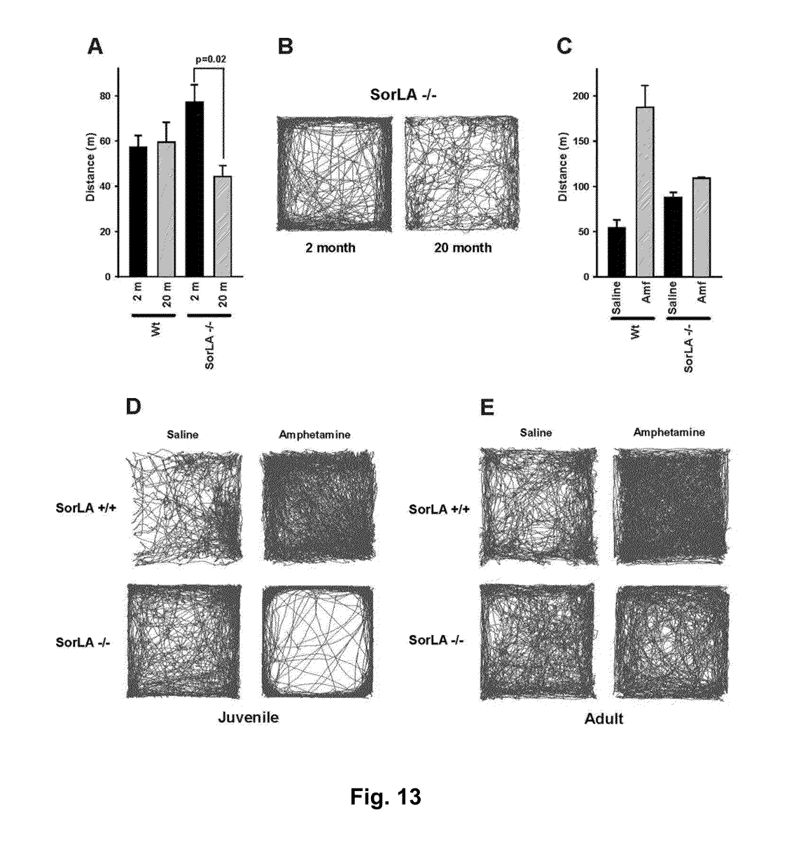

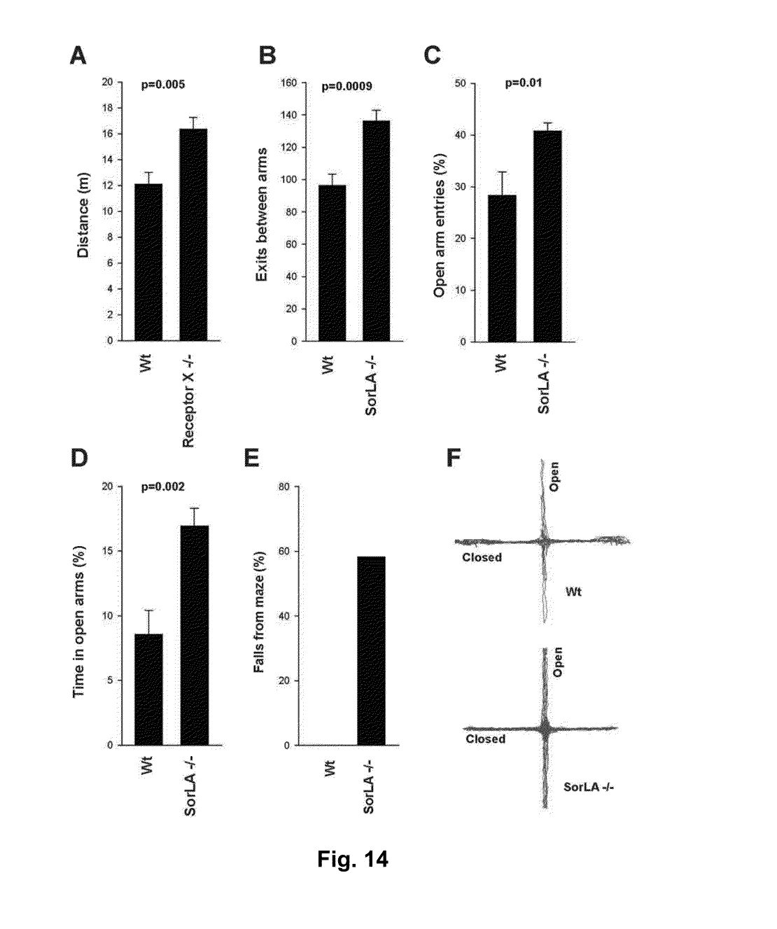

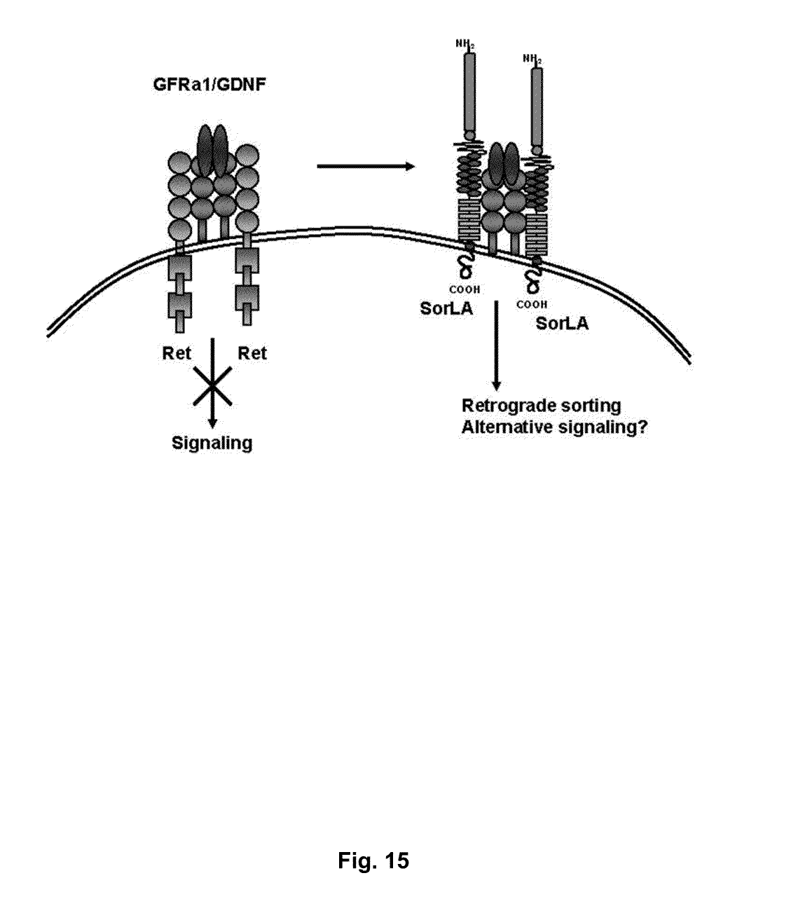

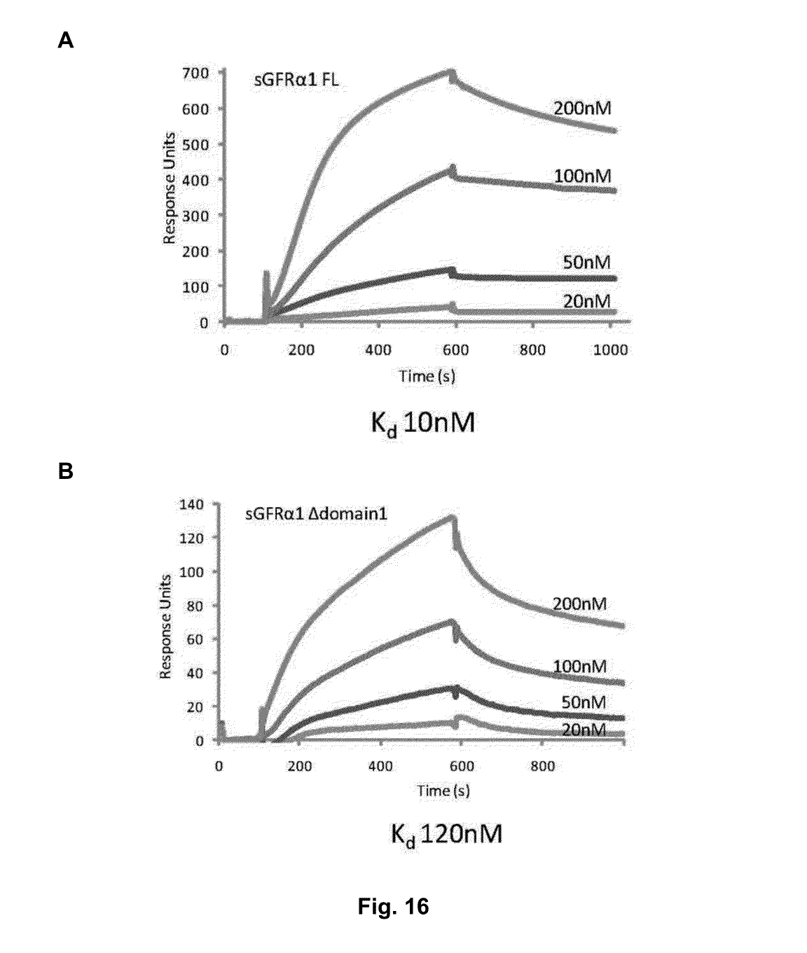

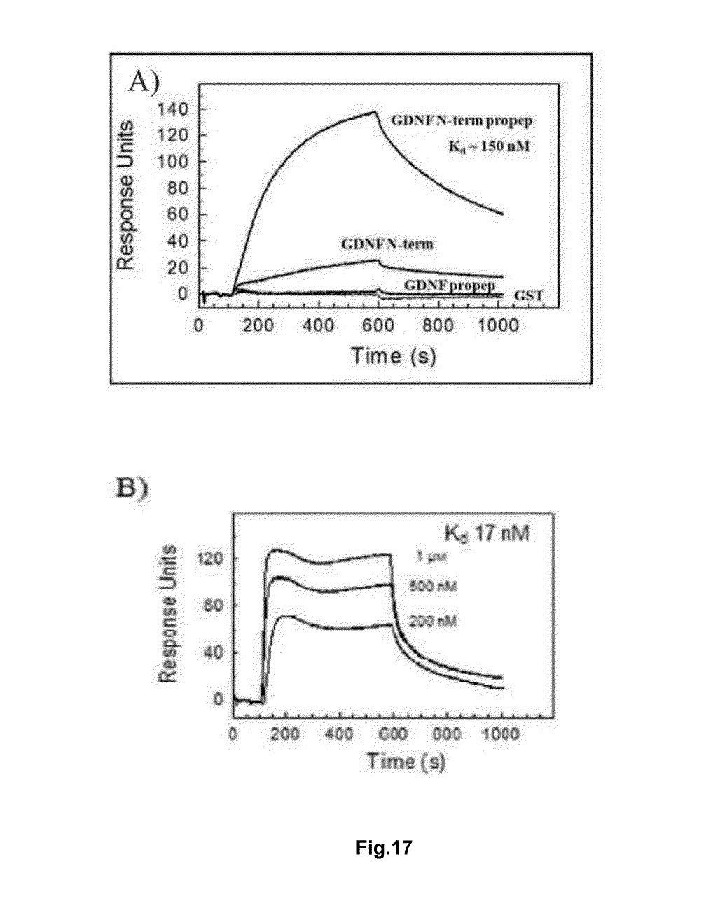

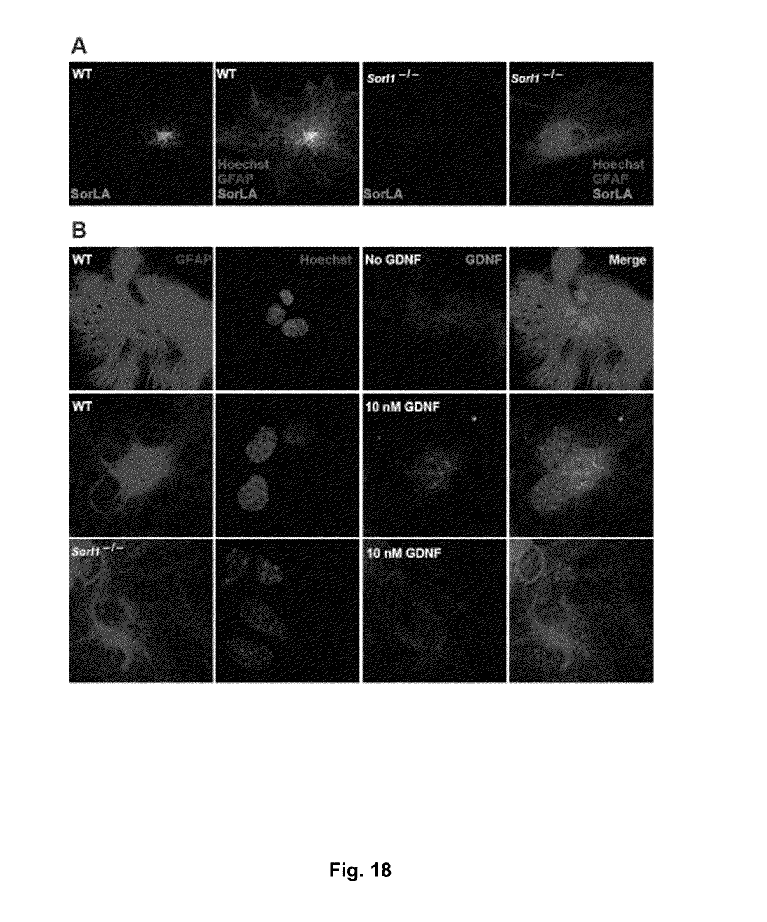

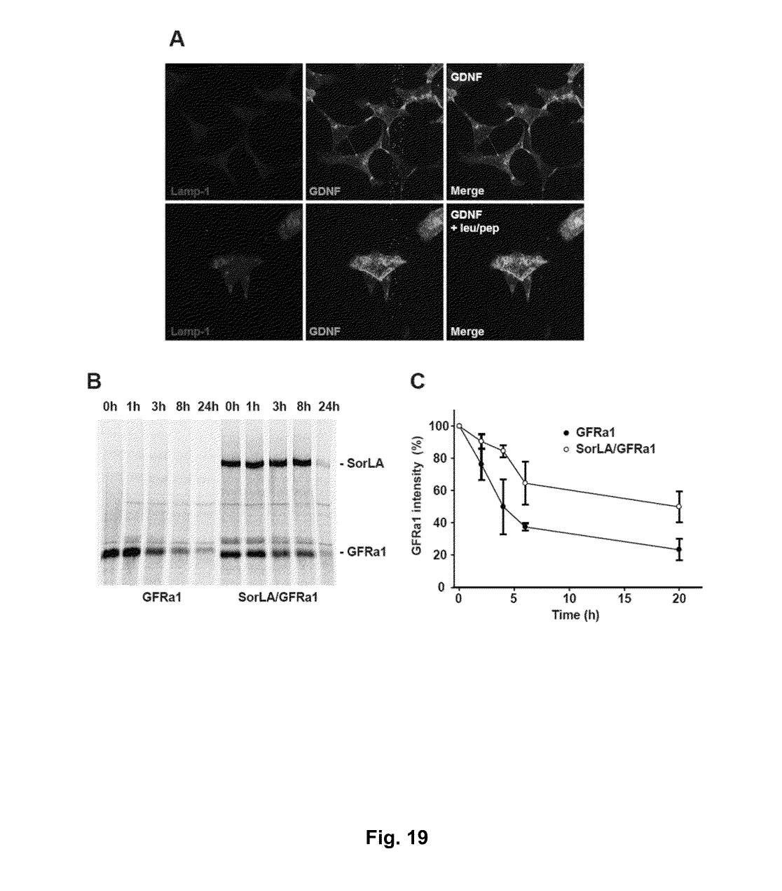

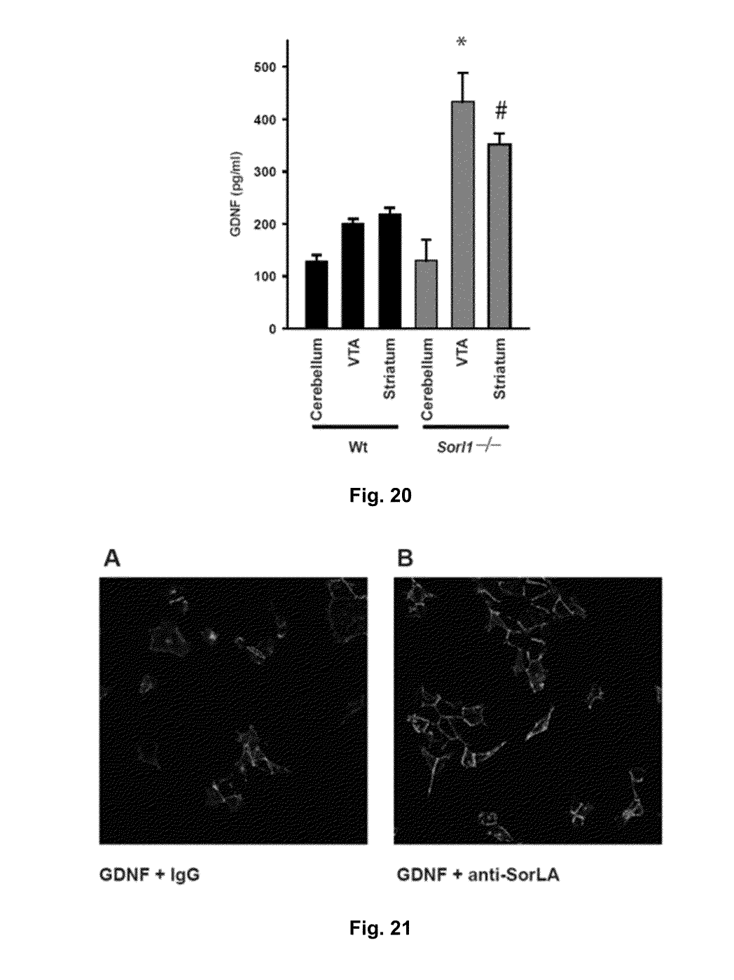

HEK293 cells stably transfected with plasmids encoding SorLA and GFRα1 were crosslinked with DSP (Pierce) and subsequently lysed. The cell lysate was incubated with antibody against SorLA or GFRα1 (R&D Systems) bound to Gammabind beads (GE Healthcare). Precipitated complexes were eluted from the washed beads with SDS loading buffer. Western blot analysis revealed the presence of a SorLA: GFRα1 complex (FIG. 5A). The direct interaction of the extracellular domains of SorLA and GFRα1 was also demonstrated using surface plasmon resonance (Biacore, Sweden) using CaHBS as standard running buffer (10 mM HEPES, pH 7.4, 140 mM NaCl, 2 mM CaCl₂, 1 mM EGTA, 0.005% Tween20). A biosensor chip from Biacore (CM5, cat. no. BR-1000-14) was activated using the NHS/EDC method as described by the supplier followed by coating with SorLA (FIG. 5C).

### Example 2

### Demonstration of a SorLA:GFRα2 complex

HEK293 cells stably transfected with plasmids encoding SorLA and GFRα2 were crosslinked with DSP (Pierce) and subsequently lysed. The cell lysate was incubated with antibody against GFRα2 (R&D Systems) bound to Gammabind beads (GE Healthcare). Precipitated complexes were eluted from the washed beads with SDS loading buffer. Western blot analysis revealed the presence of a SorLA: GFRα2 complex (FIG. 11A). The direct interaction of the extracellular domains of SorLA and GFRα2 was also demonstrated using surface plasmon resonance (Biacore, Sweden) using CaHBS as standard running buffer (10 mM HEPES, pH 7.4, 140 mM NaCl, 2 mM CaCl₂, 1 mM EGTA, 0.005% Tween-20). A biosensor chip from Biacore (CM5, cat. no. BR-1000-14) was activated using the NHS/EDC method as described by the supplier followed by coating with SorLA.

### Example 3

### Demonstration of a SorLA:GFRα3 Complex

HEK293 cells stably transfected with plasmids encoding SorLA and GFRα3 were crosslinked with DSP (Pierce) and subsequently lysed. The cell lysate was incubated with antibody against GFRα3 (R&D Systems) bound to Gammabind beads (GE Healthcare). Precipitated complexes were eluted from the washed beads with SDS loading buffer. Western blot analysis revealed the presence of a SorLA: GFRα3 complex (FIG. 11A). The direct interaction of the extracellular domains of SorLA and GFRα3 was also demonstrated using surface plasmon resonance (Biacore, Sweden) using CaHBS as standard running buffer (10 mM HEPES, pH 7.4, 140 mM NaCl, 2 mM CaCl₂, 1 mM EGTA, 0.005% Tween-20). A biosensor chip from Biacore (CM5, cat. no. BR-1000-14) was activated using the NHS/EDC method as described by the supplier followed by coating with SorLA.

### Example 4

### Demonstration of a SorLA:GFRα4 Complex

HEK293 cells stably transfected with plasmids encoding SorLA and GFRα4 were crosslinked with DSP (Pierce) and subsequently lysed. The cell lysate was incubated with antibody against GFRα4 (R&D Systems) bound to Gammabind beads (GE Healthcare). Precipitated complexes were eluted from the washed beads with SDS loading buffer. Western blot analysis revealed the presence of a SorLA: GFRα4 complex (FIG. 11A). The direct interaction of the extracellular domains of SorLA and GFRα4 was also demonstrated using surface plasmon resonance (Biacore, Sweden) using CaHBS as standard running buffer (10 mM HEPES, pH 7.4, 140 mM NaCl, 2 mM CaCl₂, 1 mM EGTA, 0.005% Tween-20). A biosensor chip from Biacore (CM5, cat. no. BR-1000-14) was activated using the NHS/EDC method as described by the supplier followed by coating with SorLA.

### Example 5

### Demonstration of a SorLA:GDNF Complex

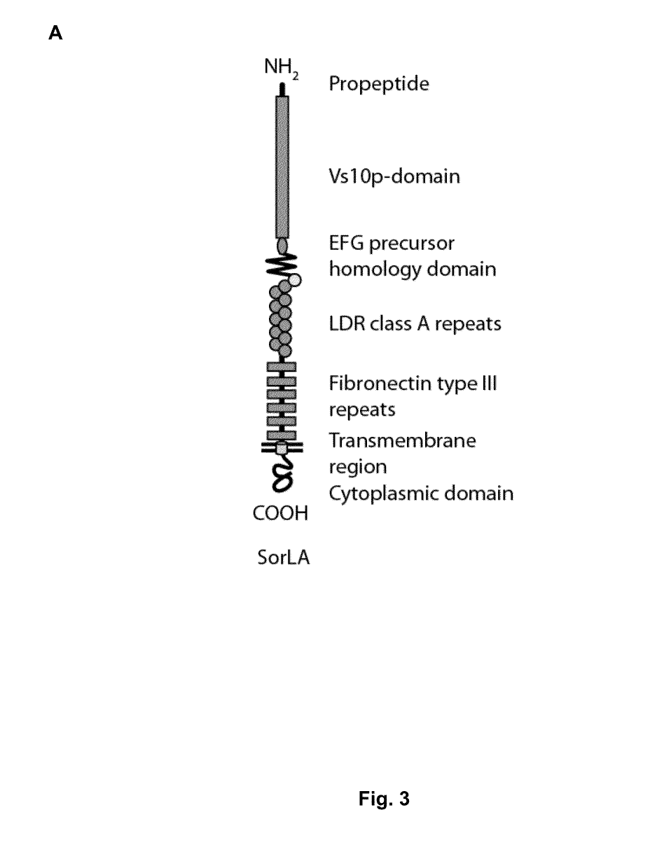

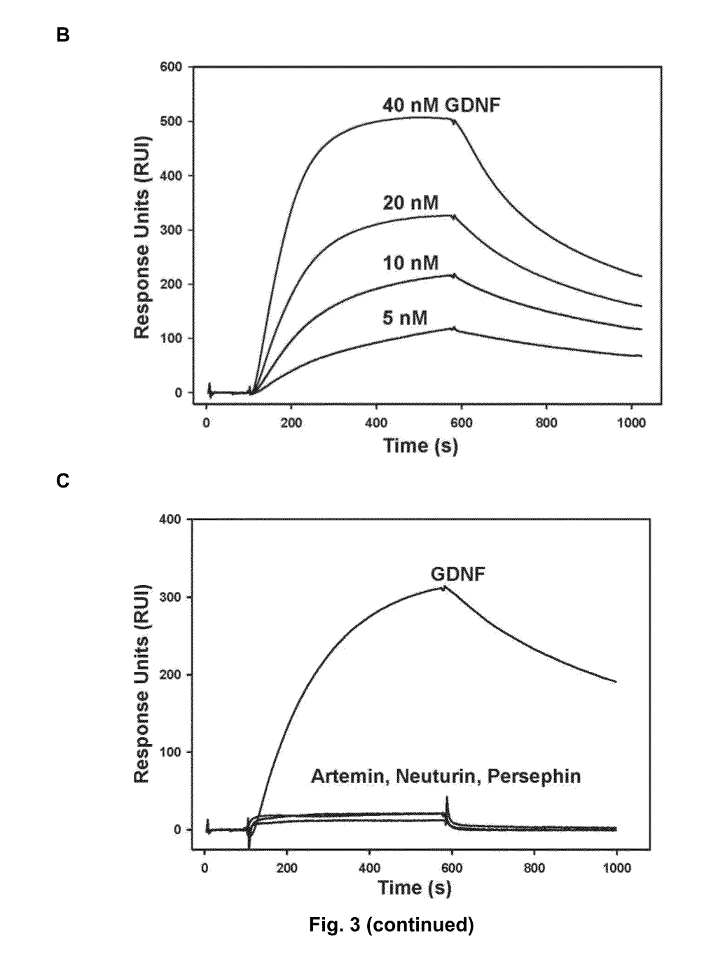

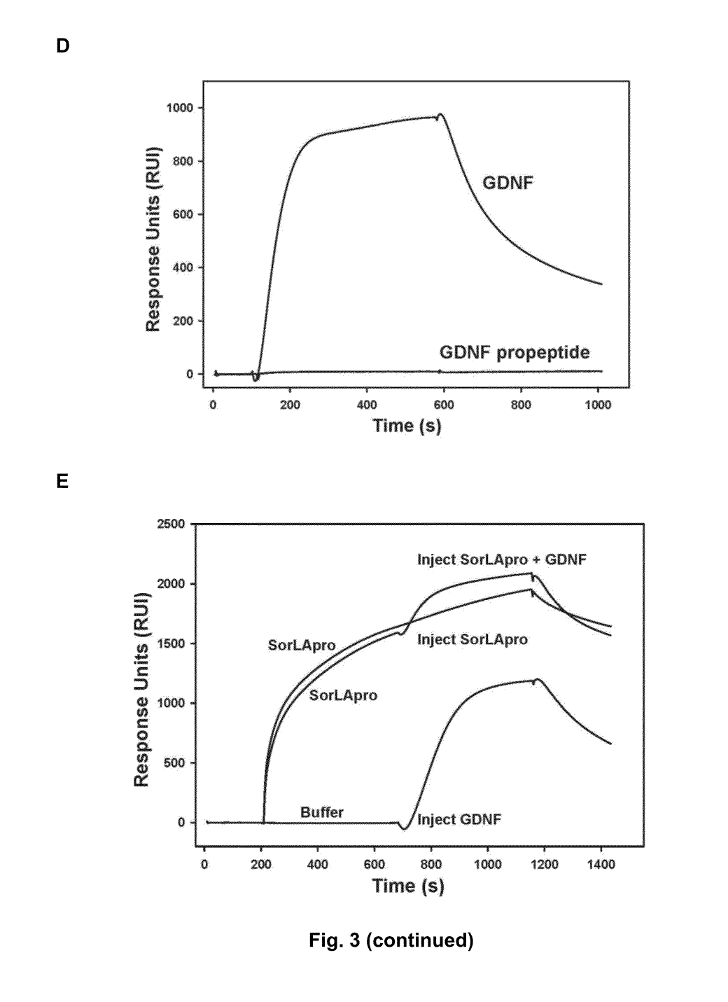

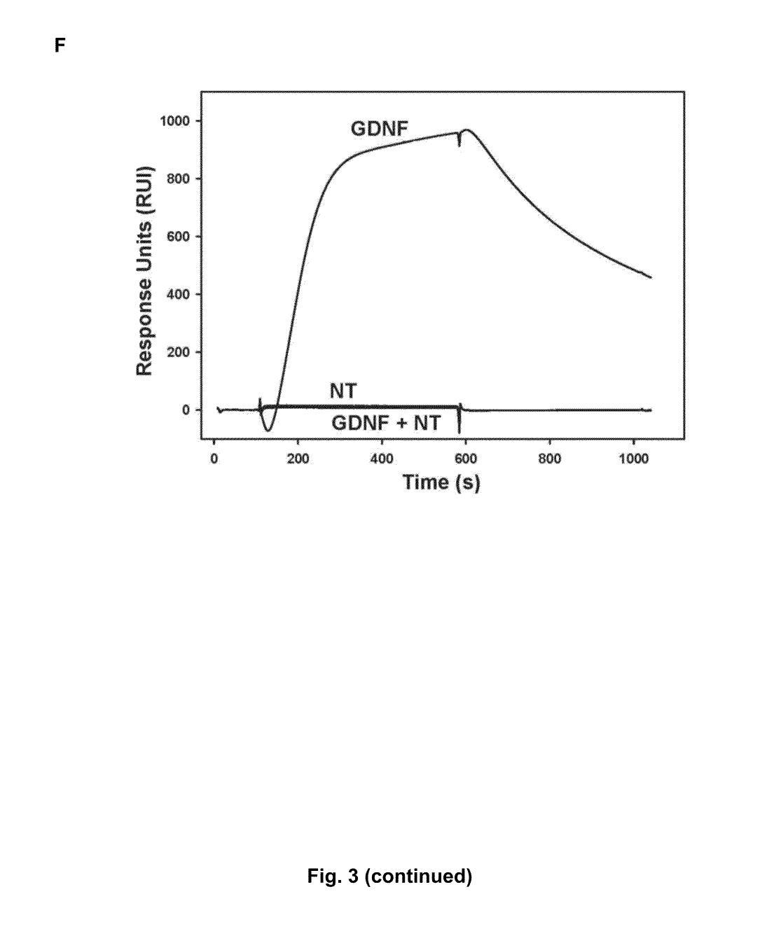

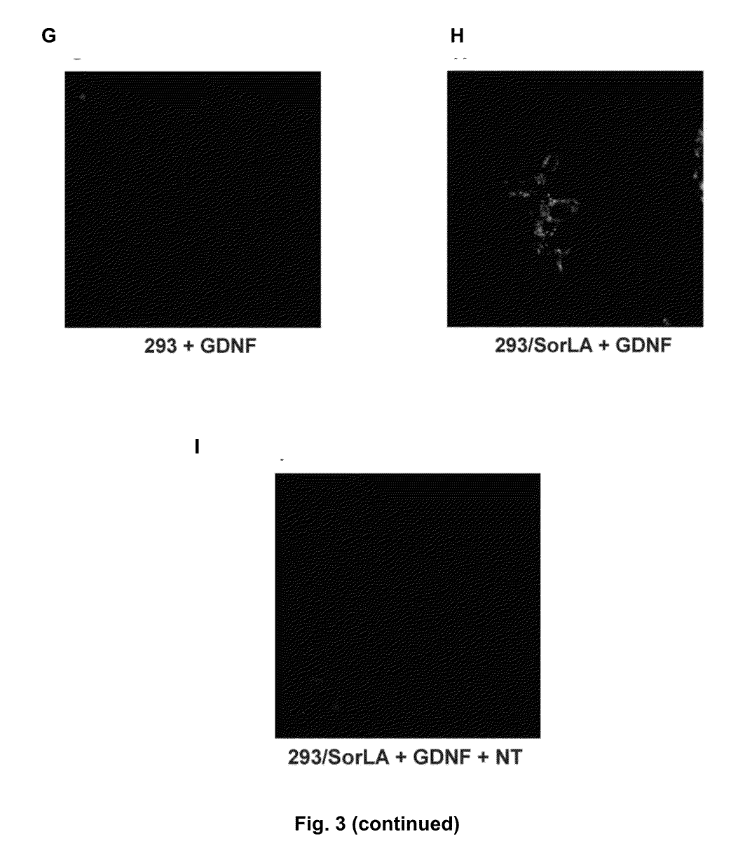

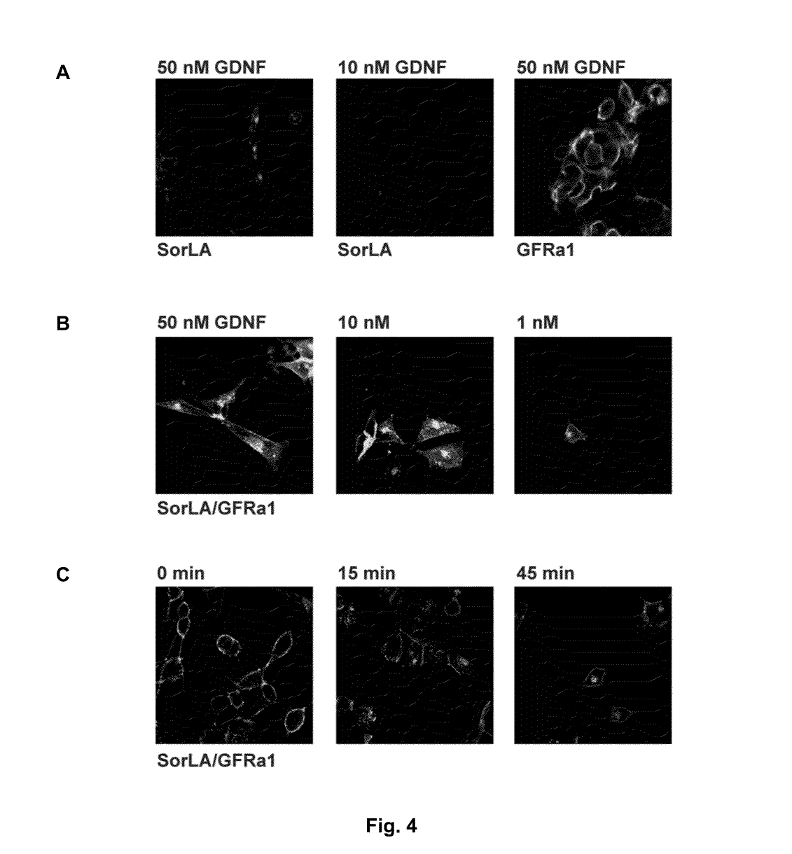

The direct interaction of the extracellular domains of SorLA and GDNF was demonstrated using surface plasmon resonance (Biacore, Sweden) using CaHBS as standard running buffer (10 mM HEPES, pH 7.4, 140 mM NaCl, 2 mM CaCl₂, 1 mM EGTA, 0.005% Tween-20). A biosensor chip from Biacore (CM5, cat. no. BR1000-14) was activated using the NHS/EDC method as described by the supplier followed by coating with SorLA (FIG. 3).

### Example 6

A cell based screening method for identifying receptor antagonists/agonists that modulates the by complexes comprising SorLA and GFRα1. Determination of binding, internalization or signaling by members of the Vps10p domain receptor family can be performed in cellular systems. Cells expressing SorLA and GFRα1 and GDNF following e.g. transfection with plasmids encoding all three receptors, respectively, are incubated with a candidate agent (inhibitor/antagonist) compound. Said agent can e.g. represent an antibody against either one of the receptors, SorLA and GFRα1, binding ligands such as the SorLA propeptide or, fragments of the respective receptors. After incubation, the cells are washed, protein complexes crosslinked with dithiobis[succinimidylpropionate] (DSP, Pierce) and subsequently lysed in THE buffer (10 mM Tris-HCl, 150 mM NaCl, 1 mM EDTA, 1% nonides P-40, pH. 8) containing proteinase inhibitors (Complete, Roche Applied Science, Switzerland). The cell lysate is incubated with antibody against SorLA or GFRα1 (R&D Systems) bound to sepharose beads (GE Healthcare). Precipitated complexes are eluted from the washed beads (acidic buffer and subsequent neutralization). Western blot analysis of SorLA and GFRα1 in the eluate reveals whether candidate compounds are able to inhibit SorLA:GFRα1 complex formation.

### Example 7

### An In Vitro Assay for Identifying Agents Disrupting the Interaction of GFRα1 and/or GDNF with SorLA

Determination of direct binding of a ligand such as a small organic molecule, a peptide or a soluble receptor including but not limited to SorLA, GFRα1 and GDNF, to immobilized protein can be performed by e.g. surface plasmon resonance analysis (Biacore, Sweden) using CaHBS as standard running buffer (10 mM HEPES, pH 7.4, 140 mM NaCl, 2 mM CaCl₂, 1 mM EGTA, and 0.005% Tween-20). Such an agent could be derived from the N-terminal of mature GDNF (FIG. 17) or the GFRα1 domain 1 (FIG. 16). A biosensor chip from Biacore (CM5, cat. no. BR-1000-14) is activated using the NHS/EDC method as described by supplier followed by coating with SorLA. Several different approaches can be applied: Candidate agents can be identified by comparing the binding signal (response units) to a chip immobilized with one of the receptors and comparing this signal to an empty flow cell. In another approach, inhibition of an established ligand can be monitored in the absence or presence of putative inhibitors. The difference in the signal depicts the inhibitory potential of the antagonist. The data collected are analysed by fitting of sensorgrams for affinity estimations and inhibitory potential using the Biaevaluation version 3.1 program. The surface Plasmon resonance assay can easily be transform into other assays in which the Vps10p-domain receptor, the ligand or the putative inhibitor is immobilized on a solid phase. For instance, receptors can be immobilized in e.g. Maxisorp microtiter wells from Nunc (cat. no. 439454) by incubation for 16 h at 4° C. in 50 mM NaHCO₃, pH 9.6. After blocking using 5% bovine serum albumin (Sigma, cat. no. A9647) for 2 h at room temperature, the wells are washed three times with MB buffer (10 mM HEPES, pH 7.4, 140 mM NaCl, 2 20 mM CaCl₂, and 1 mM MgCl₂) before incubation with a labelled ligand (e.g. iodinated) in the absence or presence of a various concentrations of a candidate inhibitor. Following incubation (e.g. overnight at 4° C.) and washing with MB buffer, bound radioactivity is released by adding 10% SDS. Nonspecific binding of tracer to wells coated only with bovine serum albumin is determined and subtracted from the values determined in the binding experiments. The binding data point can be fitted to binding equations using the Prism software from GraphPad, version 4. Likewise, the antagonist can be labelled and binding to the immobilized receptor directly measured. In yet another setup, the receptor, ligand or antagonist can be immobilized on scintillation beads and binding measured in a scintillation proximity assay in which the receptor-binding molecule has been labelled using radioactivity.

### Example 8

### A Cell Based Screening Method for Identifying Agents Capable of Inhibiting SorLA

An antagonist directed against an entity of the SorLA:GFRα1:GDNF receptor complex may act as an inhibitor of the entire complex. Accordingly it is relevant to screen for agents capable of binding to e.g. the Vps10p-domain receptor entity. Such a method is described in the present example. Determination of binding, internalization or signalling by members of the Vps10pdomain receptor family can be performed in cellular systems. Cells expressing one of the receptors, either endogenously or following e.g. transfection with a plasmid containing the cDNA of the receptor, are incubated with a radio-labeled ligand, in the absence and the presence respectively, of a candidate inhibitor/antagonist compound. After incubation, the cells are washed to remove unspecific binding and subsequently harvested. The degree of binding of the candidate antagonist/inhibitor to the receptor is determined by using a conventional radioligand assay well known to those skilled in the art. See e.g. Bylund and Toews (1993) Am J Physiol. 265(5 Pt 1):L421-9 entitled “Radioligand binding methods: practical guide and tips”. Likewise, endocytosis/internalization may be determined as described in Nykjaer et al (1992) FEBS 300:13- and Nielsen et al (2001) EMBO J.

### Example 9

### A Cell Based Screening Method for Identifying Agents Capable of Modulating Retrograde Sorting of a GFRα Receptor by SorLA

Surface localized GFRα receptor was labeled either by a fluorescent tag or fluorescent antibodies in cells expressing both SorLA and GFRα receptor, and incubated with a SorLA agonist/antagonist for 30 min. The amount of internalized GFRα receptor was subsequently evaluated using confocal microscopy as shown in FIG. 6 and FIG. 11.

### Example 10

### A Cell Based Screening Method for Identifying Agents Capable of Increasing Ret Phosphorylation

SY5Y neuroblastoma cells were stimulated by increasing concentrations of GDNF for defined periods of time in the absence or presence a SorLA antagonist or agonist.

Cells were lysed and the cell lysates were incubated with antibody against Ret (R&D Systems) coupled to Gammabind beads (GE Healthcare). Precipitated protein was eluted from the washed beads (acidic buffer and subsequent neutralization) and phosphorylated Ret was visualized by Western blotting using anti-phosphotyrosine (Milipore). (As shown in FIG. 9).

### Example 11

### A Cell Based Screening Method for Identifying Agents Capable of Increasing Cell Survival

SY5Y neuroblastoma cells were harvested by trypsin-EDTA treatment and thereafter resuspended in DMEM without phenol red (LONZA) containing 1% glutamax and 1% P/S (penicillin and streptomycin). The cells were plated: 20.000 cells pr. well in a 96 well plate in a final volume of 50 μl. Next day, cells were stimulated by increasing concentrations of GDNF in the absence or presence of a SorLA antagonist or agonist. The final volume after addition of GDNF etc. was 100 μl. Each kind of sample was made as quadruplets. The cells were incubated for 72 hours in their normal incubator. After the 72 hours the MultiTox-Fluor Multiplex Cytotoxicity Assay reagents (Promega) were prepared and added to the cells as directed by supporting technical literature from manufacturer. Briefly, for each sample, 100 μl of assay buffer, 0.1 μl of GF-AFC substrate, and 0.1 μl of bis-AAF-R110 substrate were mixed and added to the well. After 1 min mixing at 240 rpm on an orbital shaker (Ika® KS 260 Basic) the plate was incubated at 37° C. for 30 min. The fluorescence was thereafter measured with a Wallac VICTOR3™ 1420 Multilabel Counter (Perkin Elmer™ Lifesciences). Viability was measured at 400 nmEx/505 nmEm. The signal is directly proportional to the survival.

Another way to test the survival is to grow the cells on coverslips instead of 96-well plate. After the 72 hours the cells could be fixed in 4% paraformaldehyde (PFA) in PBS for 30 min at room temperature and mounted onto slides using mounting medium containing DAPI. The DAPI stained cells could be counted directly in a fluorescence microscope.

(As shown in FIG. 9D).

### Example 12

### Modulation of GDNF-Family Ligand Activities in Primary Neuronal Cell Cultures by a SorLA Antagonist/Agonist

Primary neuronal cultures are prepared from brains of wild-type mice.

At postnatal day 0-2 brains are dissected out, cells are dissociated and plated in the presence of for example 10 ng/ml GDNF, and in the presence or absence of a SorLA antagonist or agonist. Following maturation of the cultures of 1-2 weeks in vitro, the number of surviving neurons was scored.

### Example 13

### Modulation of GDNF Activity in Primary Dopaminergic Neurons by a SorLA Antagonist/Agonist

Primary dopaminergic neurons prepared from the ventral tegmental area of P0-P2 rats. The rat pups were decapitated, the brain isolated and put into cold PBS. The brain was placed ventral side down. The initial cut was through the entire brain caudal to the midbrain flexure and the second cut rostral to the flexure, the slice was laid flat. The ventral edge of the slice was cut along the top of the hypothalamus, the next cut was approximately halfway between the ventral edge of the slice and the ventricle hole. The tissue was cut into smaller segments and placed in cold L15 media (Gibco, Invitrogen). The tissue was incubated with warm papain solution (L15 media, 2 mM EDTA, 20 units/ml papain (TMWorthington, Medinova), 0.5 mM kynurenic acid and NaOH (to adjust pH to approximately 7) for 30 min. at 37° C. Media was removed and tissue was disintegrated in neuron media (Neurobasal™ media without L-glutamine (Gibco, Invitrogen), FBS, B-27 (Invitrogen), glutamax (Invitrogen), primocin (Amaxa), 5-fluorodeoxyuridine (Sigma) and Uridine (Sigma)) by gently trituration. Neurons were spun down (800 rpm, 5 min) and the pellet was diluted in neuron media and plated on a layer of cortical glia cells in the presence of 10 ng/ml GDNF and in the presence or absence of a SorLA antagonist or agonist. Neurons were incubated at 37° C. for 1 week in vitro whereafter they were fixed with 4% paraformaldehyde (PFA) in PBS for 30 min at room temperature. Thereafter they were washed twice for 15 min in PBS containing 0.1% Triton X-100, followed by incubation with 10% FBS in PBS for 20 min. Coverslips were hereafter incubated with anti-tyrosine hydroxylase antibodies (Pel freeze, 1:1000) in 10% FBS in PBS overnight at 4° C. Next day, the coverslips were washed three times in PBS containing 0.1% Triton X-100 and incubated with Alexa-Fluor® 488 goat anti-rabbit IgG (Invitrogen, 1:1000) in 10% FBS in PBS overnight at 4° C. The coverslips were thereafter washed 2×15 min in 0.1% Triton X-100 in PBS, 1×15 min in PBS and 1×15 min in water. The coverslips were mounted onto slides with Dako Flurescent Mounting Medium (Dako, Denmark). The number of surviving neurons was scored by counting the number of tyrosine hydroxylase positive neurons (as shown in FIG. 12).

### Example 14

### The Effect of a SorLA Antagonist/Agonist on Amphetamine-Induced Hyperactivity

Mice were pretreated for 1-30 days with a SorLA antagonist or agonist, and tested for amphetamine-induced hyperactivity in an open field test consisting of a (40×40×35 cm) clear Plexiglas arena. The arena was set up in a dim room under a video camera connected to a computer under the control of the Any-maze tracking system. Mice were placed in the corner of the arena and their activity was recorded over a 40 min session as shown in FIG. 13. Amphetamine (0.1-10 mg/kg) was administered intraperitoneally or intravenously.

### Example 15

### The Effect of a SorLA Antagonist/Agonist on Anxiety-Related Behavior

The behavior of wild-type and SorLA transgenic mice were tested for anxiety related behavior in an elevated plus maze. The elevated plus maze is used as an experimental model for depressive, manic, and anxiety-related behavior. Treatment of mice with antidepressive or anxiolytic agents normally increase the distance traveled, the number of line crossings, and the number of entries and time spent in the open arms. The elevated plus maze was raised 40 cm above the floor, and consisted of two opposite enclosed arms with 15 cm high opaque walls and two opposite open arms of the same size (35×5 cm). The elevated plus maze was set up in a dim lit room under a video camera connected to a computer under the control of the Any-maze tracking system. Testing sessions of 10 min were carried out for each mouse and measured the number of entries and the time spent in the open arms as described in FIG. 14. A similar experiment is performed where mice were pretreated for 1-30 days with a SorLA antagonist or agonist.

### Example 16

### The Effect of a SorLA Antagonist/Agonist on Amphetamine-Induced Sensitization

Adult mice were pretreated with 4 mg/kg amphetamine or saline, once every other day for 5 days while also being treated with a SorLA antagonist or agonist, and tested for locomotor activity in an open field. Treatment with SorLA antagonist/agonist continued for one week after the last amphetamine pretreatment. On day 16, a test for sensitization was conducted wherein all mice received a challenge injection of amphetamine (2 mg/kg) and tested for locomotor activity in the open field.

### Example 17

### The Protective Effect of a SorLA Antagonist/Agonist for MPTP-Induced Neurotoxicity in Mice

Adult mice were injected with 20 mg/kg MPTP (1-methyl-4-phenyl-1,2,3,6-tetrahydropyridine, a agent selectively toxic to dopaminergic neurons) daily for 4 days with or without a SorLA antagonist/agonist. Motor activity was recorded, 1 day prior to injections and two days after cessation of injections in an open field test consisting of a (40×40×35 cm) clear Plexiglas arena. The arena was set up in a dim room under a video camera connected to a computer under the control of the Any-maze tracking system. Mice were placed in the corner of the arena and their activity was recorded over a 60 min session. Finally, mice were perfused with 4% paraformaldehyde for assessment of various morphological markers such as tyrosine hydroxylase and dopamine transporter using immunohistochemistry.

### Example 18

### A Cell Based Screening Method for Identifying Agents Capable of Increasing Cell Differentiation

Neuroblastoma cells were harvested with trypsin-EDTA treatment. Cells were resuspended in DMEM-F12 media containing 10% FBS and 1% P/S (penicillin and streptomycin) and seeded in 6 well plate. The following day, the media was removed and the cells were incubated with fresh media and increasing concentrations of GDNF in the absence or presence of a SorLA antagonist or agonist. The cells were incubated for 72 hours in their normal incubator, where after the cells were visualized with a stereo-microscope (Leica DC300) which were connected to a camera. The neurite outgrowths were scored by defining a neurite as a process twice as long as the diameter of the cell.

### Example 19

### Methods of Treatment

The resulting developed active agent of peptide/polypeptide nature (possible antibody based) either freeze-dried to be dissolved before use or as a ready to use solution so that it can be given for parenteral administration route (e.g. intravenously (I.V.), intramuscularly (I.M.) or subcutaneously (S.C.). Mucosal application of a solid dose form also represents a possibility in this case. If the resulting developed active agent is of chemical nature a formulation for oral administration as well as a potential route is prepared e.g. for S.C. or I.M. use. The developed medicament will either be used for prophylactic purpose or given chronically for long life treatment. In this case the active agent interferes with and thereby prevents the process of molecular events to take place that leads to the symptoms of the mental and behavioural disorders. In case at that time a genetic test is developed to diagnose individuals predisposed to develop mental and behavioural disorders the medicament should be used were possible in connection with such a diagnostics. Have a mental and behavioural disorder developed; chronic treatment with the medicament represents another possibility. The rationale is constantly to be able to suppress the molecular events leading to the symptoms of the disease. Finally it will be possible to co-administer the medicament together with conventional treatments for neurological or mental and behavioural disorders e.g. with antipsychotics, antidepressants or lithium.

### Example 20

A cell based screening method for identifying agents capable of modulating internalization and degradation of a GDNF family ligand (GFL) by SorLA.

GDNF or another GFL was incubated for between 2-120 min in the presence or absence of a SorLA agonist/antagonist (e.g. an antibody) with cells expressing both SorLA and GFRα receptor. GDNF or other GFLs were labeled either by a fluorescent tag or fluorescent antibodies. The amount of internalized GDNF or GFL was subsequently evaluated using confocal microscopy as shown in FIG. 6 and FIG. 11 and FIG. 21.

### SEQUENCE LISTINGS

### SEQ ID NO 1: Homo Sapiens SorLA Polypeptide

MATRSSRRESRLPFLFTLVALLPPGALCEVWTQRLHGGSAPLPQDR
GFLVVQGDPRELRLWARGDARGASRADEKPLRRKRSAALQPEPIKV
YGQVSLNDSHNQMVVHWAGEKSNVIVALARDSLALARPKSSDVYVS
YDYGKSFKKISDKLNFGLGNRSEAVIAQFYHSPADNKRYIFADAYA
QYLWITFDFCNTLQGFSIPFRAADLLLHSKASNLLLGFDRSHPNKQ
LWKSDDFGQTWIMIQEHVKSFSWGIDPYDKPNTIYIERHEPSGYST
VFRSTDFFQSRENQEVILEEVRDFQLRDKYMFATKVVHLLGSEQQS
SVQLWVSFGRKPMRAAQFVTRHPINEYYIADASEDQVFVCVSHSNN
RTNLYISEAEGLKFSLSLENVLYYSPGGAGSDTLVRYFANEPFADF
HRVEGLQGVYIATLINGSMNEENMRSVITFDKGGTWEFLQAPAFTG
YGEKINCELSQGCSLHLAQRLSQLLNLQLRRMPILSKESAPGLIIA
TGSVGKNLASKTNVYISSSAGARWREALPGPHYYTWGDHGGIITAI
AQGMETNELKYSTNEGETWKTFIFSEKPVFVYGLLTEPGEKSTVFT
IFGSNKENVHSWLILQVNATDALGVPCTENDYKLWSPSDERGNECL
LGHKTVFKRRTPHATCFNGEDFDRPVVVSNCSCTREDYECDFGFKM
SEDLSLEVCVPDPEFSGKSYSPPVPCPVGSTYRRTRGYRKISGDTC
SGGDVEARLEGELVPCPLAEENEFILYAVRKSIYRYDLASGATEQL
PLTGLRAAVALDFDYEHNCLYWSDLALDVIQRLCLNGSTGQEVIIN
SGLETVEALAFEPLSQLLYWVDAGFKKIEVANPDGDFRLTIVNSSV
LDRPRALVLVPQEGVMFWTDWGDLKPGIYRSNMDGSAAYHLVSEDV
KWPNGISVDDQWIYWTDAYLECIERITFSGQQRSVILDNLPHPYAI
AVFKNEIYWDDWSQLSIFRASKYSGSQMEILANQLTGLMDMKIFYK
GKNTGSNACVPRPCSLLCLPKANNSRSCRCPEDVSSSVLPSGDLMC
DCPQGYQLKNNTCVKQENTCLRNQYRCSNGNCINSIWWCDFDNDCG
DMSDERNCPTTICDLDTQFRCQESGTCIPLSYKCDLEDDCGDNSDE
SHCEMHQCRSDEYNCSSGMCIRSSWVCDGDNDCRDWSDEANCTAIY
HTCEASNFQCRNGHCIPQRWACDGDTDCQDGSDEDPVNCEKKCNGF
RCPNGTCIPSSKHCDGLRDCSDGSDEQHCEPLCTHFMDFVCKNRQQ
CLFHSMVCDGIIQCRDGSDEDAAFAGCSQDPEFHKVCDEFGFQCQN
GVCISLIWKCDGMDDCGDYSDEANCENPTEAPNCSRYFQFRCENGH
CIPNRWKCDRENDCGDWSDEKDCGDSHILPFSTPGPSTCLPNYYRC
SSGTCVMDTWVCDGYRDCADGSDEEACPLLANVTAASTPTQLGRCD
RFEFECHQPKTCIPNWKRCDGHQDCQDGRDEANCPTHSTLTCMSRE
FQCEDGEACIVLSERCDGFLDCSDESDEKACSDELTVYKVQNLQWT
ADFSGDVTLTWMRPKKMPSASCVYNVYYRVVGESIWKTLETHSNKT
NTVLKVLKPDTTYQVKVQVQCLSKAHNTNDFVTLRTPEGLPDAPRN
LQLSLPREAEGVIVGHWAPPIHTHGLIREYIVEYSRSGSKMWASQR
AASNFTEIKNLLVNTLYTVRVAAVTSRGIGNWSDSKSITTIKGKVI
PPPDIHIDSYGENYLSFTLTMESDIKVNGYVVNLFWAFDTHKQERR
TLNFRGSILSHKVGNLTAHTSYEISAWAKTDLGDSPLAFEHVMTRG
VRPPAPSLKAKAINQTAVECTWTGPRNVVYGIFYATSFLDLYRNPK
SLTTSLHNKTVIVSKDEQYLFLVRVVVPYQGPSSDYVVVKMIPDSR
LPPRHLHVVHTGKTSVVIKWESPYDSPDQDLLYAVAVKDLIRKTDR
SYKVKSRNSTVEYTLNKLEPGGKYHIIVQLGNMSKDSSIKITTVSL
SAPDALKIITENDHVLLFWKSLALKEKHFNESRGYEIHMFDSAMNI
TAYLGNTTDNFFKISNLKMGHNYTFTVQARCLFGNQICGEPAILLY
DELGSGADASATQAARSTDVAAVVVPILFLILLSLGVGFAILYTKH
RRLQSSFTAFANSHYSSRLGSAIFSSGDDLGEDDEDAPMITGFSDD
VPMVIA

### SEQ ID NO 2: Homo Sapiens SorLA Propetide

EVWTQRLHGGSAPLPQDRGFLVVQGDPRELRLWARGDARGASRADE
KPLRRKR

### SEQ ID NO 3: Homo Sapiens SorLA Vps10p-Domain

SAALQPEPIKVYGQVSLNDSHNQMVVHWAGEKSNVIVALARDSLAL
ARPKSSDVYVSYDYGKSFKKISDKLNFGLGNRSEAVIAQFYHSPAD
NKRYIFADAYAQYLWITFDFCNTLQGFSIPFRAADLLLHSKASNLL
LGFDRSHPNKQLWKSDDFGQTWIMIQEHVKSFSWGIDPYDKPNTIY
IERHEPSGYSTVFRSTDFFQSRENQEVILEEVRDFQLRDKYMFATK
VVHLLGSEQQSSVQLWVSFGRKPMRAAQFVTRHPINEYYIADASED
QVFVCVSHSNNRTNLYISEAEGLKFSLSLENVLYYSPGGAGSDTLV
RYFANEPFADFHRVEGLQGVYIATLINGSMNEENMRSVITFDKGGT
WEFLQAPAFTGYGEKINCELSQGCSLHLAQRLSQLLNLQLRRMPIL
SKESAPGLIIATGSVGKNLASKTNVYISSSAGARWREALPGPHYYT
WGDHGGIITAIAQGMETNELKYSTNEGETWKTFIFSEKPVFVYGLL
TEPGEKSTVFTIFGSNKENVHSWLILQVNATDALGVPCTENDYKLW
SPSDERGNECLLGHKTVFKRRTPHATCFNGEDFDRPVVVSNCSCTR
EDYECDFGFKMSEDLSLEVCVPDPEFSGKSYSPPVPCPVGSTYRRT
RGYRKISGDTCSGGDVEARLEGELVPCPLAEE

### SEQ ID NO 4: Homo Sapiens GFRα1 Polypeptide Isoform a

MFLATLYFALPLLDLLLSAEVSGGDRLDCVKASDQCLKEQSCSTKY
RTLRQCVAGKETNFSLASGLEAKDECRSAMEALKQKSLYNCRCKRG
MKKEKNCLRIYWSMYQSLQGNDLLEDSPYEPVNSRLSDIFRVVPFI
SDVFQQVEHIPKGNNCLDAAKACNLDDICKKYRSAYITPCTTSVSN
DVCNRRKCHKALRQFFDKVPAKHSYGMLFCSCRDIACTERRRQTIV
PVCSYEEREKPNCLNLQDSCKTNYICRSRLADFFTNCQPESRSVSS
CLKENYADCLLAYSGLIGTVMTPNYIDSSSLSVAPWCDCSNSGNDL
EECLKFLNFFKDNTCLKNAIQAFGNGSDVTVWQPAFPVQTTTATTT
TALRVKNKPLGPAGSENEIPTHVLPPCANLQAQKLKSNVSGNTHLC
ISNGNYEKEGLGASSHITTKSMAAPPSCGLSPLLVLVVTALSTLLS
LTETS

### SEQ ID NO 5: Homo Sapiens GFRα1 Polypeptide Isoform a, Theoretically Binding Site to SorLA

DRLDCVKASDQCLKEQSCSTKYRTLRQCVAGKETNFSLASGLEAKD
ECRSAMEALKQKSLYNCRCKRGMKKEKNCLRIYWSMYQSLQGNDLL
EDSPYEPVNSRLSDIFRVVPFISDVFQQ

### SEQ ID NO 6: Homo Sapiens GFRα1 Polypeptide Isoform b

MFLATLYFALPLLDLLLSAEVSGGDRLDCVKASDQCLKEQSCSTKY
RTLRQCVAGKETNFSLASGLEAKDECRSAMEALKQKSLYNCRCKRG
MKKEKNCLRIYWSMYQSLQGNDLLEDSPYEPVNSRLSDIFRVVPFI
SVEHIPKGNNCLDAAKACNLDDICKKYRSAYITPCTTSVSNDVCNR
RKCHKALRQFFDKVPAKHSYGMLFCSCRDIACTERRRQTIVPVCSY
EEREKPNCLNLQDSCKTNYICRSRLADFFTNCQPESRSVSSCLKEN
YADCLLAYSGLIGTVMTPNYIDSSSLSVAPWCDCSNSGNDLEECLK
FLNFFKDNTCLKNAIQAFGNGSDVTVWQPAFPVQTTTATTTTALRV
KNKPLGPAGSENEIPTHVLPPCANLQAQKLKSNVSGNTHLCISNGN
YEKEGLGASSHITTKSMAAPPSCGLSPLLVLVVTALSTLLSLTETS

### SEQ ID NO 7: Homo Sapiens GFRα1 Polypeptide Isoform b, Theoretically Binding Site to SorLA

DRLDCVKASDQCLKEQSCSTKYRTLRQCVAGKETNFSLASGLEAKD
ECRSAMEALKQKSLYNCRCKRGMKKEKNCLRIYWSMYQSLQGNDLL
EDSPYEPVNSRLSDIFRVVPFIS

### SEQ ID NO 8: Homo Sapiens GFRα2 Polypeptide

MILANVFCLFFFLDETLRSLASPSSLQGPELHGWRPPVDCVRANEL
CAAESNCSSRYRTLRQCLAGRDRNTMLANKECQAALEVLQESPLYD
CRCKRGMKKELQCLQIYWSIHLGLTEGEEFYEASPYEPVTSRLSDI
FRLASIFSGTGADPVVSAKSNHCLDAAKACNLNDNCKKLRSSYISI
CNREISPTERCNRRKCHKALRQFFDRVPSEYTYRMLFCSCQDQACA
ERRRQTILPSCSYEDKEKPNCLDLRGVCRTDHLCRSRLADFHANCR
ASYQTVTSCPADNYQACLGSYAGMIGFDMTPNYVDSSPTGIVVSPW
CSCRGSGNMEEECEKFLRDFTENPCLRNAIQAFGNGTDVNVSPKGP
SFQATQAPRVEKTPSLPDDLSDSTSLGTSVITTCTSVQEQGLKANN
SKELSMCFTELTTNIIPGSNKVIKPNSGPSRARPSAALTVLSVLML
KLAL

### SEQ ID NO 9: Homo Sapiens GFRα2 Polypeptide, Short Isoform

MILANVFCLFFFLGTGADPVVSAKSNHCLDAAKACNLNDNCKKLRS
SYISICNREISPTERCNRRKCHKALRQFFDRVPSEYTYRMLFCSCQ
DQACAERRRQTILPSCSYEDKEKPNCLDLRGVCRTDHLCRSRLADF
HANCRASYQTVTSCPADNYQACLGSYAGMIGFDMTPNYVDSSPTGI
VVSPWCSCRGSGNMEEECEKFLRDFTENPCLRNAIQAFGNGTDVNV
SPKGPSFQATQAPRVEKTPSLPDDLSDSTSLGTSVITTCTSVQEQG
LKANNSKELSMCFTELTTNIIPGSNKVIKPNSGPSRARPSAALTVL
SVLMLKLAL

### SEQ ID NO 10: Homo Sapiens GFRα3 Polypeptide

MVRPLNPRPLPPVVLMLLLLLPPSPLPLAAGDPLPTESRLMNSCLQ
ARRKCQADPTCSAAYHHLDSCTSSISTPLPSEEPSVPADCLEAAQQ
LRNSSLIGCMCHRRMKNQVACLDIYWTVHRARSLGNYELDVSPYED
TVTSKPWKMNLSKLNMLKPDSDLCLKFAMLCTLNDKCDRLRKAYGE
ACSGPHCQRHVCLRQLLTFFEKAAEPHAQGLLLCPCAPNDRGCGER
RRNTIAPNCALPPVAPNCLELRRLCFSDPLCRSRLVDFQTHCHPMD
ILGTCATEQSRCLRAYLGLIGTAMTPNFVSNVNTSVALSCTCRGSG
NLQEECEMLEGFFSHNPCLTEAIAAKMRFHSQLFSQDWPHPTFAVM
AHQNENPAVRPQPWVPSLFSCTLPLILLLSLW

### SEQ ID NO 11: Homo Sapiens GFRα3 Polypeptide, Isoform 2

MVRPLNPRPLPPVVLMLLLLLPPSPLPLAAGDPLPTESRLMNSCLQ
ARRKCQADPTCSAAYHHLDSCTSSISTPLPSEEPSVPADCLEAAQQ
LRNSSLIGCMCHRRMKNQVACLDIYWTVHRARSLDSDLCLKFAMLC
TLNDKCDRLRKAYGEACSGPHCQRHVCLRQLLTFFEKAAEPHAQGL
LLCPCAPNDRGCGERRRNTIAPNCALPPVAPNCLELRRLCFSDPLC
RSRLVDFQTHCHPMDILGTCATEQSRCLRAYLGLIGTAMTPNFVSN
VNTSVALSCTCRGSGNLQEECEMLEGFFSHNPCLTEAIAAKMRFHS
QLFSQDWPHPTFAVMAHQNENPAVRPQPWVPSLFSCTLPLILLLSL
W

### SEQ ID NO 12: Homo Sapiens GFRα4 Polypeptide

MVRCLGPALLLLLLLGSASSVGGNRCVDAAEACTADARCQRLRSEY
VAQCLGRAAQGGCPRARCRRALRRFFARGPPALTHALLFCPCAGPA
CAERRRQTFVPSCAFSGPGPAPPSCLEPLNFCERSRVCRCARAAAG
PWRGWGRGLSPAHRPPAAQASPPGLSGLVHPSAQRPRRLPAGPGRP
LPARLRGPRGVPAGTAVTPNYVDNVSARVAPWCDCGASGNRREDCE
AFRGLFTRNRCLDGAIQAFASGWPPVLLDQLNPQGDPEHSLLQVSS
TGRALERRSLLSILPVLALPALL

### SEQ ID NO 13: Homo Sapiens Neurotensin Polypeptide

QLYENKPRRPYIL

### SEQ ID NO 14: Homo Sapiens Neurotensin Polypeptide Theoretically Binding Site

YIL

### SEQ ID NO 15: Homo Sapiens Neurotensin Polypeptide Theoretically Binding Site

PYIL

### SEQ ID NO 16: Homo Sapiens GDNF Polypeptide

MKLWDVVAVCLVLLHTASAFPLPAGKRPPEAPAEDRSLGRRRAPFA
LSSDSNMPEDYPDQFDDVMDFIQATIKRLKRSPDKQMAVLPRRERN
RQAAAANPENSRGKGRRGQRGKNRGCVLTAIHLNVTDLGLGYETKE
ELIFRYCSGSCDAAETTYDKILKNLSRNRRLVSDKVGQACCRPIAF
DDDLSFLDDNLVYHILRKHSAKRCGCI

### SEQ ID NO 17: Homo Sapiens GDNF Polypeptide, Propeptide

FPLPAGKRPPEAPAEDRSLGRRRAPFALSSDSNMPEDYPDQFDDVM
DFIQATIKRLKR

### SEQ ID NO 18: Homo Sapiens GDNF Polypeptide, Bindingsite

SPDKQMAVLPRRERNRQAAAANPENSRGKGRRGQRGKN

1. A method of treating attention deficit and hyperactivity disorder (ADHD), wherein said method comprises administering an antibody that binds Sorting Protein-Related Receptor (SorLA) and antagonizes interaction between SorLA and Glia cell-derived neurotrophic factor receptor al (GFRα1) to a patient in need thereof, to thereby increase extracellular levels of said Glia cell-derived neurotrophic factor (GDNF) in the brain of said patient and treat said attention deficit and hyperactivity disorder (ADHD).

2. A method of treating an anxiety disorder, wherein said method comprises administering an antibody that binds Sorting Protein-Related Receptor (SorLA) and antagonizes interaction between SorLA and Glia cell-derived neurotrophic factor receptor al (GFRα1) to a patient in need thereof, to thereby increase extracellular levels of said Glia cell-derived neurotrophic factor (GDNF) in the brain of said patient and treat said anxiety disorder.

3. A method of treating drug abuse, wherein said method comprises administering an antibody that binds Sorting Protein-Related Receptor (SorLA) and antagonizes interaction between SorLA and Glia cell-derived neurotrophic factor receptor al (GFRα1) to a patient in need thereof, to thereby increase extracellular levels of said Glia cell-derived neurotrophic factor (GDNF) in the brain of said patient and treat said drug abuse.

4. The method of claim 1, wherein said antibody binds a binding site on an extracellular domain of SorLA.

5. The method of claim 4, wherein said binding site comprises SEQ ID NO:3, or has at least 80% sequence identity to SEQ ID NO:3.

6. The method of claim 1, wherein said antibody is a polyclonal antibody, a monoclonal antibody, a humanized antibody, chimeric antibody, or single-chain antibody.

7. The method according to claim 1, wherein said antibody is isolated or recombinant.

8. The method of claim 2, wherein said antibody binds a binding site on an extracellular domain of SorLA.

9. The method of claim 8, wherein said binding site comprises SEQ ID NO:3, or has at least 80% sequence identity to SEQ ID NO:3.

10. The method of claim 2, wherein said antibody is a polyclonal antibody, a monoclonal antibody, a humanized antibody, chimeric antibody, or single-chain antibody.

11. The method according to claim 2, wherein said antibody is isolated or recombinant.

12. The method of claim 3, wherein said antibody binds a binding site on an extracellular domain of SorLA.

13. The method of claim 12, wherein said binding site comprises SEQ ID NO:3, or has at least 80% sequence identity to SEQ ID NO:3.

14. The method of claim 3, wherein said antibody is a polyclonal antibody, a monoclonal antibody, a humanized antibody, chimeric antibody, or single-chain antibody.

15. The method according to claim 3, wherein said antibody is isolated or recombinant.
